SEQUENCE LISTING (2 sequences)

SEQ ID NO: 1 - RNA, 19 nt, synthetic construct
  source 1..19: mol_type = other RNA
  modified_base order(1,3,5,7,9,11,13,15,17,19): mod_base = OTHER; note = 2- prime-O-methyl ribonucleotide
  modified_base order(2,4,6,8,10,12,14,16,18): mod_base = OTHER; note = 2-prime-fluoro-ribonucleotide
  modified_base order(1^2,2^3,17^18,18^19): mod_base = OTHER; note = Phosphorothioate linkage between adjacent nucleotides
ataactctgtccattaccg

SEQ ID NO: 2 - RNA, 19 nt, synthetic construct
  source 1..19: mol_type = other RNA
  modified_base order(1..6,10..19): mod_base = OTHER; note = 2- prime-O-methyl ribonucleotide
  modified_base order(7,8,9): mod_base = OTHER; note = 2-prime-fluoro-ribonucleotide
  modified_base order(17^18,18^19): mod_base = OTHER; note = Phosphorothioate linkage between adjacent nucleotides
cggtaatggacagagttat

(19) United States
(12) Patent Application Publication
(10) Pub. No.: US 2025/0163415 A1
(43) Pub. Date: May 22, 2025

(54) Therapeutic Inhibition of LPA Expression

(71) Applicant: SILENCE THERAPEUTICS GMBH, Berlin (DE)

(72) Inventors: David Rider, Berlin (DE); Daniel Swerdlow, London (GB); Giles Camplon, London (GB); Alison Scrimgeour, London (GB); Curtis Rambaran, London (GB); Rosamund Wilson, Malborough Wiltshire (GB); Matthias Machacek, Basel (CH); Pascal Schulthess, Basel (CH); James Wade, Basel (CH)

(21) Appl. No.: 18/837,507

(22) PCT Filed: Feb. 8, 2023
(86) PCT No.: PCT/EP2023/053136

§ 371 Date: Aug. 9, 2024

Related U.S. Application Data
(60) Provisional application No. 63/420,195, filed on Oct. 28, 2022.
(60) Provisional application No. 63/395,246, filed on Aug. 4, 2022.
(60) Provisional application No. 63/320,381, filed on Mar. 16, 2022.
(60) Provisional application No. 63/308,167, filed on Feb. 9, 2022.

(51) Int. Cl. C12N 15/113; A61P 3/06; A61P 9/10

(52) U.S. Cl. CPC C12N 15/113; A61P 3/06; A61P 9/10; C12N 2310/14; C12N 2310/351

(57) ABSTRACT

The invention relates to the therapeutic inhibition of LPA gene expression, and in particular to effective doses and dosage regimes for the therapeutic use of the SLN360 molecule, in the prophylaxis or treatment of conditions associated with elevated levels of Lp(a). Such conditions include stroke, atherosclerosis, thrombosis, and cardiovascular diseases such as coronary heart disease and aortic stenosis.

### FIELD OF THE INVENTION

The present invention relates to the therapeutic inhibition of LPA gene expression, and in particular to effective doses and dosage regimes for the therapeutic use of the SLN360 molecule, in the prophylaxis or treatment of conditions associated with elevated levels of Lp(a). Such conditions include stroke, atherosclerosis, thrombosis, and cardiovascular diseases such as coronary heart disease and aortic stenosis.

### BACKGROUND

Lipoprotein(a) [Lp(a)] has long been recognized as a genetically-determined, independent risk factor for atherothrombotic cardiovascular disease.¹ Mendelian randomization studies consistently demonstrate a causal relationship between plasma concentrations of Lp(a) and the risk of myocardial infarction, stroke, peripheral arterial disease, and cardiovascular death.² Patients with Lp(a) concentrations in the highest decile (approximately 350 nmol/L) have a life-time risk of cardiovascular disease equivalent to familial hypercholesterolemia.³⁻⁴ Elevated levels of Lp(a) are also associated with aortic valve calcification, the development and more rapid progression of aortic stenosis, and a higher rate of aortic valve replacement.⁵⁻⁶ Although some therapies have moderate Lp(a) lowering effects, such as administration of niacin or PCSK9-inhibitors, currently no drug treatments for elevated Lp(a) concentrations have received regulatory approval.

The LPA gene encodes for apolipoprotein(a), [apo(a)], a dominant and a rate-limiting component in the hepatic synthesis of the Lp(a) particle.⁷ Efforts have been underway for several years to develop RNA-targeted therapies that reduce hepatic production of apo(a) and thereby reduce plasma concentrations of Lp(a). Previous phase 1 and 2 studies have demonstrated that an antisense oligonucleotide (ASO), pelacarsen, can reduce Lp(a) plasma concentrations by up to 80% with weekly administration.⁸

SLN360 is a short-interfering RNA (siRNA) to target LPA messenger RNA. SLN360 is a 19-mer siRNA covalently linked to a tri-antennary N-acetyl-galactosamine (GalNAc) moiety. The GalNAc conjugate binds to asialoglycoprotein receptors, which are expressed almost exclusively on hepatocytes. This approach results in selective uptake and concentration of SLN360 in hepatocytes enabling the drug to bind and degrade the messenger RNA that encodes for apo(a), described in WO 2020/099476.

### SUMMARY OF THE INVENTION

In a first aspect, the invention provides SLN360 for use in the prophylaxis or treatment of a condition associated with elevated levels of Lp(a), wherein said SLN360 is for administration in an individual dose of 100 mg to 600 mg.

In a further aspect, the invention provides a method of prophylaxis or treatment of a condition associated with elevated levels of Lp(a) in a subject in need thereof, wherein said SLN360 is administered in an individual dose of 100 mg to 600 mg.

In a further aspect, the invention provides the use of SLN360 in the preparation of a medicament or pharmaceutical composition for the prophylaxis or treatment of a condition associated with elevated levels of Lp(a), wherein said SLN360 is for administration in an individual dose of 100 mg to 600 mg.

Thus, in all aspects, an individual dose may provide SLN360 in an amount of, for example:
100-600 mg, 125-600 mg, 150-600 mg, 175-600 mg, 200-600 mg, 225-600 mg, 250-600 mg, 275-600 mg, 300-600 mg, 325-600 mg, 350-600 mg, 375-600 mg, 400-600 mg, 425-600 mg, 450-600 mg, 475-600 mg, 500-600 mg, 525-600 mg, 550-600 mg or 575-600 mg;
100-575 mg, 125-575 mg, 150-575 mg, 175-575 mg, 200-575 mg, 225-575 mg, 250-575 mg, 275-575 mg, 300-575 mg, 325-575 mg, 350-575 mg, 375-575 mg, 400-575 mg, 425-575 mg, 450-575 mg, 475-575 mg, 500-575 mg, 525-575 mg or 550-575 mg;
100-550 mg, 125-550 mg, 150-550 mg, 175-550 mg, 200-550 mg, 225-550 mg, 250-550 mg, 275-550 mg, 300-550 mg, 325-550 mg, 350-550 mg, 375-550 mg, 400-550 mg, 425-550 mg, 450-550 mg, 475-550 mg, 500-550 mg or 525-550 mg;
100-525 mg, 125-525 mg, 150-525 mg, 175-525 mg, 200-525 mg, 225-525 mg, 250-525 mg, 275-525 mg, 300-525 mg, 325-525 mg, 350-525 mg, 375-525 mg, 400-525 mg, 425-525 mg, 450-525 mg, 475-525 mg or 500-525 mg;
100-500 mg, 125-500 mg, 150-500 mg, 175-500 mg, 200-500 mg, 225-500 mg, 250-500 mg, 275-500 mg, 300-500 mg, 325-500 mg, 350-500 mg, 375-500 mg, 400-500 mg, 425-500 mg, 450-500 mg or 475-500 mg;
100-475 mg, 125-475 mg, 150-475 mg, 175-475 mg, 200-475 mg, 225-475 mg, 250-475 mg, 275-475 mg, 300-475 mg, 325-475 mg, 350-475 mg, 375-475 mg, 400-475 mg, 425-475 mg or 450-475 mg;
100-450 mg, 125-450 mg, 150-450 mg, 175-450 mg, 200-450 mg, 225-450 mg, 250-450 mg, 275-450 mg, 300-450 mg, 325-450 mg, 350-450 mg, 375-450 mg, 400-450 mg or 425-450 mg;
100-425 mg, 125-425 mg, 150-425 mg, 175-425 mg, 200-425 mg, 225-425 mg, 250-425 mg, 275-425 mg, 300-425 mg, 325-425 mg, 350-425 mg, 375-425 mg or 400-425 mg;
100-400 mg, 125-400 mg, 150-400 mg, 175-400 mg, 200-400 mg, 225-400 mg, 250-400 mg, 275-400 mg, 300-400 mg, 325-400 mg, 350-400 mg or 375-400 mg;
100-375 mg, 125-375 mg, 150-375 mg, 175-375 mg, 200-375 mg, 225-375 mg, 250-375 mg, 275-375 mg, 300-375 mg, 325-375 mg or 350-375 mg;
100-350 mg, 125-350 mg, 150-350 mg, 175-350 mg, 200-350 mg, 225-350 mg, 250-350 mg, 275-350 mg, 300-350 mg or 325-350 mg;
100-325 mg, 125-325 mg, 150-325 mg, 175-325 mg, 200-325 mg, 225-325 mg, 250-325 mg, 275-325 mg or 300-325 mg;
100-300 mg, 125-300 mg, 150-300 mg, 175-300 mg, 200-300 mg, 225-300 mg, 250-300 mg or 275-300 mg;
100-275 mg, 125-275 mg, 150-275 mg, 175-275 mg, 200-275 mg, 225-275 mg or 250-275 mg;
100-250 mg, 125-250 mg, 150-250 mg, 175-250 mg, 200-250 mg or 225-250 mg;
100-225 mg, 125-225 mg, 150-225 mg, 175-225 mg or 200-225 mg;
100-200 mg, 125-200 mg, 150-200 mg or 175-200 mg;
100-175 mg, 125-175 mg or 150-175 mg;
100-150 mg or 125-150 mg; or
100-125 mg.

It may be desirable for an individual dose to provide SLN360 in an amount of 300 mg to 600 mg, for example:
300-600 mg, 325-600 mg, 350-600 mg, 375-600 mg, 400-600 mg, 425-600 mg, 450-600 mg, 475-600 mg, 500-600 mg, 525-600 mg, 550-600 mg or 575-600 mg;
300-575 mg, 325-575 mg, 350-575 mg, 375-575 mg, 400-575 mg, 425-575 mg, 450-575 mg, 475-575 mg, 500-575 mg, 525-575 mg or 550-575 mg;
300-550 mg, 325-550 mg, 350-550 mg, 375-550 mg, 400-550 mg, 425-550 mg, 450-550 mg, 475-550 mg, 500-550 mg or 525-550 mg;
300-525 mg, 325-525 mg, 350-525 mg, 375-525 mg, 400-525 mg, 425-525 mg, 450-525 mg, 475-525 mg or 500-525 mg;
300-500 mg, 325-500 mg, 350-500 mg, 375-500 mg, 400-500 mg, 425-500 mg, 450-500 mg or 475-500 mg;
300-475 mg, 325-475 mg, 350-475 mg, 375-475 mg, 400-475 mg, 425-475 mg or 450-475 mg;
300-450 mg, 325-450 mg, 350-450 mg, 375-450 mg, 400-450 mg or 425-450 mg;
300-425 mg, 325-425 mg, 350-425 mg, 375-425 mg or 400-425 mg;
300-400 mg, 325-400 mg, 350-400 mg or 375-400 mg;
300-375 mg, 325-375 mg or 350-375 mg;
300-350 mg or 325-350 mg; or
300-325 mg.

For example, an individual dose may provide SLN360 in an amount of about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg or about 600 mg.

The term “about” in relation to a numerical value should be taken to mean+/−10%, e.g., +/−5%.

Thus, an individual dose may provide SLN360 in an amount of 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg or 600 mg.

In certain embodiments, an individual dose preferably provides SLN360 in an amount of 300 mg or in an amount of 450 mg.

It will be understood that the amount of SLN360 to be administered in an individual dosage is typically independent of the body weight of the recipient subject.

It may be desirable that SLN360 is administered to a subject via a dosing regime comprising a plurality or course of doses.

Typically, administration under the dosing regime will be once monthly, or less frequent, e.g., 2-monthly (once every 2 months), 3-monthly (once every 3 months), 4-monthly (once every 4 months), 5-monthly (once every 5 months), 6-monthly (once every 6 months), 7-monthly (once every 7 months), 8-monthly (once every 8 months), 9-monthly (once every 9 months), 10-monthly (once every 10 months), 11-monthly (once every 11 months) or 12-monthly (once every 12 months) administration.

In certain embodiments, administration under the dosing regime is preferably 4-monthly or 6-monthly.

In certain embodiments, an individual dose preferably provides SLN360 in an amount of 300 mg and administration under the dosing regime is 4-monthly.

In certain embodiments, an individual dose preferably provides SLN360 in an amount of 300 mg and administration under the dosing regime is 6-monthly.

In certain embodiments, an individual dose preferably provides SLN360 in an amount of 450 mg and administration under the dosing regime is 6-monthly.

Thus the interval between consecutive doses will typically be one or more months. For example, the interval between doses may be at least 1 month, at least 2 months, at least 3 months, at least 4 months, at least 5 months, at least 6 months, at least 7 months, at least 8 months, at least 9 months, at least 10 months, at least 11 months, or at least 12 months.

In certain embodiments, the interval may preferably be at least 4 months or at least 6 months.

In certain embodiments, an individual dose provides SLN360 preferably in an amount of 300 mg and the interval between consecutive doses is preferably at least 4 months.

In certain embodiments, an individual dose preferably provides SLN360 in an amount of 300 mg and the interval between consecutive doses is preferably at least 6 months.

In certain embodiments, an individual dose preferably provides SLN360 in an amount of 450 mg and the interval between consecutive doses is preferably at least 6 months.

For example, the interval may be 1 month, 2 months, 3 months, 4 months, 5 months, 6 months, 7 months, 8 months, 9 months, 10 months, 11 months, or 12 months.

In certain embodiments, the interval may preferably be 4 months or 6 months.

In certain embodiments, an individual dose preferably provides SLN360 in an amount of 300 mg and the interval between consecutive doses is preferably 4 months.

In certain embodiments, an individual dose preferably provides SLN360 in an amount of 300 mg and the interval between consecutive doses is preferably 6 months.

In certain embodiments, an individual dose preferably provides SLN360 in an amount of 450 mg and the interval between consecutive doses is preferably 6 months.

In the context of the present disclosure, a month may be a calendar month, or a period of 28 to 31 days, e.g., a period of 30 days. However, it will be understood that precise monthly dosing may not be practical for any individual subject. Thus, consecutive doses may be separated by the relevant number of months, plus or minus 7 days, e.g., plus or minus 5 days, e.g., plus or minus 3 days, e.g., plus or minus 2 days, e.g., plus or minus 1 day.

Thus, where monthly dosing is indicated, consecutive doses may be separated by 1 calendar month or 30 days, plus or minus 7 days, e.g., plus or minus 5 days, e.g., plus or minus 3 days, e.g., plus or minus 2 days, e.g., plus or minus 1 day;
where 2-monthly dosing is indicated, consecutive doses may be separated by 2 calendar months or 60 days, plus or minus 7 days, e.g., plus or minus 5 days, e.g., plus or minus 3 days, e.g., plus or minus 2 days, e.g., plus or minus 1 day;
where 3-monthly dosing is indicated, consecutive doses may be separated by 3 calendar months or 90 days, plus or minus 7 days, e.g., plus or minus 5 days, e.g., plus or minus 3 days, e.g., plus or minus 2 days, e.g., plus or minus 1 day;
where 4-monthly dosing is indicated, consecutive doses may be separated by 4 calendar months or 120 days, plus or minus 7 days, e.g., plus or minus 5 days, e.g., plus or minus 3 days, e.g., plus or minus 2 days, e.g., plus or minus 1 day;
where 5-monthly dosing is indicated, consecutive doses may be separated by 5 calendar months or 150 days, plus or minus 7 days, e.g., plus or minus 5 days, e.g., plus or minus 3 days, e.g., plus or minus 2 days, e.g., plus or minus 1 day;
where 6-monthly dosing is indicated, consecutive doses may be separated by 6 calendar months or 180 days, plus or minus 7 days, e.g., plus or minus 5 days, e.g., plus or minus 3 days, e.g., plus or minus 2 days, e.g., plus or minus 1 day;
where 7-monthly dosing is indicated, consecutive doses may be separated by 7 calendar months or 210 days, plus or minus 7 days, e.g., plus or minus 5 days, e.g., plus or minus 3 days, e.g., plus or minus 2 days, e.g., plus or minus 1 day;
where 8-monthly dosing is indicated, consecutive doses may be separated by 8 calendar months or 240 days, plus or minus 7 days, e.g., plus or minus 5 days, e.g., plus or minus 3 days, e.g., plus or minus 2 days, e.g., plus or minus 1 day;
where 9-monthly dosing is indicated, consecutive doses may be separated by 9 calendar months or 270 days, plus or minus 7 days, e.g., plus or minus 5 days, e.g., plus or minus 3 days, e.g., plus or minus 2 days, e.g., plus or minus 1 day;
where 10-monthly dosing is indicated, consecutive doses may be separated by 10 calendar months or 300 days, plus or minus 7 days, e.g., plus or minus 5 days, e.g., plus or minus 3 days, e.g., plus or minus 2 days, e.g., plus or minus 1 day;
where 11-monthly dosing is indicated, consecutive doses may be separated by 11 calendar months or 330 days, plus or minus 7 days, e.g., plus or minus 5 days, e.g., plus or minus 3 days, e.g., plus or minus 2 days, e.g., plus or minus 1 day;
where 12 monthly dosing is indicated, consecutive doses may be separated by 365 days or 12 calendar months, plus or minus 7 days, e.g., plus or minus 5 days, e.g., plus or minus 3 days, e.g., plus or minus 2 days, e.g., plus or minus 1 day.

In certain embodiments, SLN360 is administered to a subject via a dosing regime comprising for example a first dose, a second dose, a third dose, and optionally one or more subsequent doses.

The interval between the first two doses may be 4 weeks, while the interval between the second and third doses, and between any subsequent doses, may be 5 weeks.

The interval between the first two doses may be 4 weeks, while the interval between the second and third doses, and between any subsequent doses, may be 6 weeks.

The interval between the first two doses may be 3 weeks, while the interval between the second and third doses, and between any subsequent doses, may be 4 weeks.

The interval between the first two doses may be 3 weeks, while the interval between the second and third doses, and between any subsequent doses, may be 5 weeks.

The interval between the first two doses may be 3 weeks, while the interval between the second and third doses, and between any subsequent doses, may be 6 weeks.

The interval between the first two doses may be 2 weeks, while the interval between the second and third doses, and between any subsequent doses, may be 3 weeks.

The interval between the first two doses may be 2 weeks, while the interval between the second and third doses, and between any subsequent doses, may be 4 weeks.

The interval between the first two doses may be 2 weeks, while the interval between the second and third doses, and between any subsequent doses, may be 5 weeks.

The interval between the first two doses may be 2 weeks, while the interval between the second and third doses, and between any subsequent doses, may be 6 weeks.

The interval between the first two doses may be 1 week, while the interval between the second and third doses, and between any subsequent doses, may be 3 weeks.

The interval between the first two doses may be 1 week, while the interval between the second and third doses, and between any subsequent doses, may be 4 weeks.

The interval between the first two doses may be 1 week, while the interval between the second and third doses, and between any subsequent doses, may be 5 weeks.

The interval between the first two doses may be 1 week, while the interval between the second and third doses, and between any subsequent doses, may be 6 weeks.

In certain embodiments, the interval between the first two doses is 4 weeks, while the interval between the second and third doses, and between any subsequent doses, may be 6 weeks.

In certain embodiments, the interval between the first two doses is 3 weeks, while the interval between the second and third doses, and between any subsequent doses, may be 4 weeks.

In certain embodiments, the interval between the first two doses is 3 weeks, while the interval between the second and third doses, and between any subsequent doses, may be 6 weeks.

In certain embodiments, the interval between the first two doses is 2 weeks, while the interval between the second and third doses, and between any subsequent doses, may be 4 weeks.

In certain embodiments, the interval between the first two doses is 2 weeks, while the interval between the second and third doses, and between any subsequent doses, may be 6 weeks.

The first dose provides SLN360 in an amount of 100 mg-600 mg and the second, the third and any subsequent dose provides SLN360 in an amount of 200 mg-600 mg. In certain embodiments, the first dose provides SLN360 in an amount of 200 mg, and the second, the third and any subsequent dose provides SLN360 in an amount of 200 mg.

In certain embodiments, the first dose provides SLN360 in an amount of 200 mg, and the second, the third and any subsequent dose provides SLN360 in an amount of 300 mg.

In certain embodiments, the first dose provides SLN360 in an amount of 200 mg, and the second, the third and any subsequent dose provides SLN360 in an amount of 450 mg.

In certain embodiments, the first dose provides SLN360 in an amount of 300 mg, and the second, the third and any subsequent dose provides SLN360 in an amount of 200 mg.

In certain embodiments, the first dose provides SLN360 in an amount of 300 mg, and the second, the third and any subsequent dose provides SLN360 in an amount of 300 mg.

In certain embodiments, the first dose provides SLN360 in an amount of 300 mg, and the second, the third and any subsequent dose provides SLN360 in an amount of 450 mg.

In certain embodiments, the first dose provides SLN360 in an amount of 450 mg, and the second, the third and any subsequent dose provides SLN360 in an amount of 200 mg.

In certain embodiments, the first dose provides SLN360 in an amount of 450 mg, and the second, the third and any subsequent dose SLN360 in an amount of 300 mg.

In certain embodiments, the first dose provides SLN360 in an amount of 450 mg, and the second, the third and any subsequent dose SLN360 in an amount of 450 mg.

In certain embodiments the interval between the first two doses is 4 weeks, while the interval between the second and third doses, and between any subsequent doses is 6 weeks, wherein the first dose provides SLN360 in an amount of 200 mg and the second, the third and any subsequent dose provides SLN360 in an amount of 200 mg.

In certain embodiments the interval between the first two doses is 4 weeks, while the interval between the second and third doses, and between any subsequent doses is 6 weeks, wherein the first dose provides SLN360 in an amount of 200 mg, and the second, the third and any subsequent dose provides SLN360 in an amount of 300 mg.

In certain embodiments the interval between the first two doses is 4 weeks, while the interval between the second and third doses, and between any subsequent doses is 6 weeks, wherein the first dose provides SLN360 in an amount of 200 mg, and the second, the third and any subsequent dose provides SLN360 in an amount of 450 mg.

In certain embodiments the interval between the first two doses is 4 weeks, while the interval between the second and third doses, and between any subsequent doses is 6 weeks, wherein the first dose provides SLN360 in an amount of 300 mg, and the second, the third and any subsequent dose provides SLN360 in an amount of 200 mg.

In certain embodiments the interval between the first two doses is 4 weeks, while the interval between the second and third doses, and between any subsequent doses is 6 weeks, wherein the first dose provides SLN360 in an amount of 300 mg and the second, the third and any subsequent dose provides SLN360 in an amount of 300 mg.

In certain embodiments the interval between the first two doses is 4 weeks, while the interval between the second and third doses, and between any subsequent doses is 6 weeks, wherein the first dose provides SLN360 in an amount of 300 mg, and the second, the third and any subsequent dose provides SLN360 in an amount of 450 mg.

In certain embodiments the interval between the first two doses is 4 weeks, while the interval between the second and third doses, and between any subsequent doses is 6 weeks, wherein the first dose provides SLN360 in an amount of 450 mg, and the second, the third and any subsequent dose provides SLN360 in an amount of 200 mg.

In certain embodiments the interval between the first two doses is 4 weeks, while the interval between the second and third doses, and between any subsequent doses is 6 weeks, wherein the first dose provides SLN360 in an amount of 450 mg, and the second, the third and any subsequent dose provides SLN360 in an amount of 300 mg.

In certain embodiments the interval between the first two doses is 4 weeks, while the interval between the second and third doses, and between any subsequent doses is 6 weeks, wherein the first dose provides SLN360 in an amount of 450 mg and the second, the third and any subsequent dose provides SLN360 in an amount of 450 mg.

In certain embodiments the interval between the first two doses is 3 weeks, while the interval between the second and third doses, and between any subsequent doses is 4 weeks, wherein the first dose provides SLN360 in an amount of 200 mg and the second, the third and any subsequent dose provides SLN360 in an amount of 200 mg.

In certain embodiments the interval between the first two doses is 3 weeks, while the interval between the second and third doses, and between any subsequent doses is 4 weeks, wherein the first dose provides SLN360 in an amount of 200 mg, and the second, the third and any subsequent dose provides SLN360 in an amount of 300 mg.

In certain embodiments the interval between the first two doses is 3 weeks, while the interval between the second and third doses, and between any subsequent doses is 4 weeks, wherein the first dose provides SLN360 in an amount of 200 mg, and the second, the third and any subsequent dose provides SLN360 in an amount of 450 mg.

In certain embodiments the interval between the first two doses is 3 weeks, while the interval between the second and third doses, and between any subsequent doses is 4 weeks, wherein the first dose provides SLN360 in an amount of 300 mg, and the second, the third and any subsequent dose provides SLN360 in an amount of 200 mg.

In certain embodiments the interval between the first two doses is 3 weeks, while the interval between the second and third doses, and between any subsequent doses is 4 weeks, wherein the first dose provides SLN360 in an amount of 300 mg and the second, the third and any subsequent dose provides SLN360 in an amount of 300 mg.

In certain embodiments the interval between the first two doses is 3 weeks, while the interval between the second and third doses, and between any subsequent doses is 4 weeks, wherein the first dose provides SLN360 in an amount of 300 mg, and the second, the third and any subsequent dose provides SLN360 in an amount of 450 mg.

In certain embodiments the interval between the first two doses is 3 weeks, while the interval between the second and third doses, and between any subsequent doses is 4 weeks, wherein the first dose provides SLN360 in an amount of 450 mg, and the second, the third and any subsequent dose provides SLN360 in an amount of 200 mg.

In certain embodiments the interval between the first two doses is 3 weeks, while the interval between the second and third doses, and between any subsequent doses is 4 weeks, wherein the first dose provides SLN360 in an amount of 450 mg, and the second, the third and any subsequent dose provides SLN360 in an amount of 300 mg.

In certain embodiments the interval between the first two doses is 3 weeks, while the interval between the second and third doses, and between any subsequent doses is 4 weeks, wherein the first dose provides SLN360 in an amount of 450 mg and the second, the third and any subsequent dose provides SLN360 in an amount of 450 mg.

In certain embodiments the interval between the first two doses is 2 weeks, while the interval between the second and third doses, and between any subsequent doses is 4 weeks, wherein the first dose provides SLN360 in an amount of 200 mg, and the second, the third and any subsequent dose provides SLN360 in an amount of 200 mg.

In certain embodiments the interval between the first two doses is 2 weeks, while the interval between the second and third doses, and between any subsequent doses is 4 weeks, wherein the first dose provides SLN360 in an amount of 200 mg, and the second, the third and any subsequent dose provides SLN360 in an amount of 300 mg.

In certain embodiments the interval between the first two doses is 2 weeks, while the interval between the second and third doses, and between any subsequent doses is 4 weeks, wherein the first dose provides SLN360 in an amount of 200 mg, and the second, the third and any subsequent dose provides SLN360 in an amount of 450 mg.

In certain embodiments the interval between the first two doses is 2 weeks, while the interval between the second and third doses, and between any subsequent doses is 4 weeks, wherein the first dose provides SLN360 in an amount of 300 mg, and the second, the third and any subsequent dose provides SLN360 in an amount of 200 mg.

In certain embodiments the interval between the first two doses is 2 weeks, while the interval between the second and third doses, and between any subsequent doses is 4 weeks, wherein the first dose provides SLN360 in an amount of 300 mg, and the second, the third and any subsequent dose provides SLN360 in an amount of 300 mg.

In certain embodiments the interval between the first two doses is 2 weeks, while the interval between the second and third doses, and between any subsequent doses is 4 weeks, wherein the first dose provides SLN360 in an amount of 300 mg, and the second, the third and any subsequent dose provides SLN360 in an amount of 450 mg.

In certain embodiments the interval between the first two doses is 2 weeks, while the interval between the second and third doses, and between any subsequent doses is 4 weeks, wherein the first dose provides SLN360 in an amount of 450 mg and the second, the third and any subsequent dose provides SLN360 in an amount of 200 mg.

In certain embodiments the interval between the first two doses is 2 weeks, while the interval between the second and third doses, and between any subsequent doses is 4 weeks, wherein the first dose provides SLN360 in an amount of 450 mg, and the second, the third and any subsequent dose provides SLN360 in an amount of 300 mg.

In certain embodiments the interval between the first two doses is 2 weeks, while the interval between the second and third doses, and between any subsequent doses is 4 weeks, wherein the first dose provides SLN360 in an amount of 450 mg, and the second, the third and any subsequent dose provides SLN360 in an amount of 450 mg.

In certain embodiments the interval between the first two doses is 2 weeks, while the interval between the second and third doses, and between any subsequent doses is 6 weeks, wherein the first dose provides SLN360 in an amount of 200 mg, and the second, the third and any subsequent dose provides SLN360 in an amount of 200 mg.

In certain embodiments the interval between the first two doses is 2 weeks, while the interval between the second and third doses, and between any subsequent doses is 6 weeks, wherein the first dose provides SLN360 in an amount of 200 mg, and the second, the third and any subsequent dose provides SLN360 in an amount of 300 mg.

In certain embodiments the interval between the first two doses is 2 weeks, while the interval between the second and third doses, and between any subsequent doses is 6 weeks, wherein the first dose provides SLN360 in an amount of 200 mg, and the second, the third and any subsequent dose provides SLN360 in an amount of 450 mg.

In certain embodiments the interval between the first two doses is 2 weeks, while the interval between the second and third doses, and between any subsequent doses is 6 weeks, wherein the first dose provides SLN360 in an amount of 300 mg, and the second, the third and any subsequent dose provides SLN360 in an amount of 200 mg.

In certain embodiments the interval between the first two doses is 2 weeks, while the interval between the second and third doses, and between any subsequent doses is 6 weeks, wherein the first dose provides SLN360 in an amount of 300 mg, and the second, the third and any subsequent dose provides SLN360 in an amount of 300 mg.

In certain embodiments the interval between the first two doses is 2 weeks, while the interval between the second and third doses, and between any subsequent doses is 6 weeks, wherein the first dose provides SLN360 in an amount of 300 mg, and the second, the third and any subsequent dose provides SLN360 in an amount of 450 mg.

In certain embodiments the interval between the first two doses is 2 weeks, while the interval between the second and third doses, and between any subsequent doses is 6 weeks, wherein the first dose provides SLN360 in an amount of 450 mg, and the second, the third and any subsequent dose provides SLN360 in an amount of 200 mg.

In certain embodiments the interval between the first two doses is 2 weeks, while the interval between the second and third doses, and between any subsequent doses is 6 weeks, wherein the first dose provides SLN360 in an amount of 450 mg, and the second, the third and any subsequent dose provides SLN360 in an amount of 300 mg.

In certain embodiments the interval between the first two doses is 2 weeks, while the interval between the second and third doses, and between any subsequent doses is 6 weeks wherein the first dose provides SLN360 in an amount of 450 mg, and the second, the third and any subsequent dose provides SLN360 in an amount of 450 mg.

In certain embodiments the interval between the first two doses is 3 weeks, while the interval between the second and third doses, and between any subsequent doses is 6 weeks, wherein the first dose provides SLN360 in an amount of 200 mg, and the second, the third and any subsequent dose provides SLN360 in an amount of 200 mg.

In certain embodiments the interval between the first two doses is 3 weeks, while the interval between the second and third doses, and between any subsequent doses is 6 weeks, wherein the first dose provides SLN360 in an amount of 200 mg, and the second, the third and any subsequent dose provides SLN360 in an amount of 300 mg.

In certain embodiments the interval between the first two doses is 3 weeks, while the interval between the second and third doses, and between any subsequent doses is 6 weeks, wherein the first dose provides SLN360 in an amount of 200 mg, and the second, the third and any subsequent dose provides SLN360 in an amount of 450 mg.

In certain embodiments the interval between the first two doses is 3 weeks, while the interval between the second and third doses, and between any subsequent doses is 6 weeks, wherein the first dose provides SLN360 in an amount of 300 mg, and the second, the third and any subsequent dose provides SLN360 in an amount of 200 mg.

In certain embodiments the interval between the first two doses is 3 weeks, while the interval between the second and third doses, and between any subsequent doses is 6 weeks, wherein the first dose provides SLN360 in an amount of 300 mg, and the second, the third and any subsequent dose provides SLN360 in an amount of 300 mg.

In certain embodiments the interval between the first two doses is 3 weeks, while the interval between the second and third doses, and between any subsequent doses is 6 weeks, wherein the first dose provides SLN360 in an amount of 300 mg, and the second, the third and any subsequent dose provides SLN360 in an amount of 450 mg.

In certain embodiments the interval between the first two doses is 3 weeks, while the interval between the second and third doses, and between any subsequent doses is 6 weeks, wherein the first dose provides SLN360 in an amount of 450 mg, and the second, the third and any subsequent dose provides SLN360 in an amount of 200 mg.

In certain embodiments the interval between the first two doses is 3 weeks, while the interval between the second and third doses, and between any subsequent doses is 6 weeks, wherein the first dose provides SLN360 in an amount of 450 mg, and the second, the third and any subsequent dose provides SLN360 in an amount of 300 mg.

In certain embodiments the interval between the first two doses is 3 weeks, while the interval between the second and third doses, and between any subsequent doses is 6 weeks, wherein the first dose provides SLN360 in an amount of 450 mg, and the second, the third and any subsequent dose provides SLN360 in an amount of 450 mg.

### SLN360

SLN360 is a double stranded RNA (dsRNA) or siRNA capable of inhibiting expression of the human LPA gene. Without wishing to be bound by theory, inhibition is believed to occur by RNA interference.

### SLN360 Consists of:

### (i) A First (Antisense) Strand Having the Sequence:

mA (ps) fU (ps) mA fA mC fU mC fU mG fU mC fC mA
fU mU fA mC (ps) fC (ps) mG

and

### (ii) A Second (Sense) Strand Having the Sequence:

[M]-mC mG mG mU mA mA fU fG fG mA mC mA mG mA mG
mU mU (ps) mA (ps) mU

where [M] is a triantennary ligand moiety linked to the 5′ end of the second strand and having the structure:

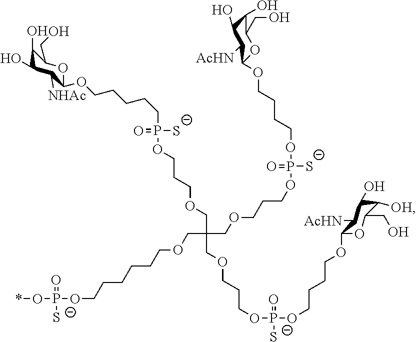

Thus, the terminal phosphorothioate group of the ligand moiety [M] is bonded directly (via the “free” bond indicated “*”) to the 5′ position of the 5′ terminal nucleotide of the second strand (i.e., to the 5′-carbon of the 2′-O-methyl ribose moiety of the mC residue at the 5′ end of the second strand).

In the context of the above formula, the following abbreviations are used:

| Abbreviation | Meaning |
|---|---|
| mA, mU, mC, mG | 2′-O-methyl ribonucleotide |
| | (adenine, uracil, cytosine, guanine) |
| fA, fU, fC, fG | 2′-fluoro-ribonucleotide |
| | (adenine, uracil, cytosine, guanine) |
| | [Interchangeable with |
| | 2′-fluoro-2′-deoxyribonucleotide.] |
| (ps) | phosphorothioate linkage |
| | between adjacent nucleotides |

Where no group is specifically indicated between adjacent nucleotides, they are linked by a conventional phosphodiester linkage. Thus, the first strand contains phosphorothioate linkages between each of the three terminal nucleotides at the 5′ end and each of the three terminal nucleotides at the 3′ end (i.e., 2 phosphorothioate linkages at each end of the strand). The other nucleotides of the first strand are linked by phosphodiester linkages. The second strand contains phosphorothioate linkages between each of the three terminal nucleotides at the 3′ end (i.e., 2 phosphorothioate linkages at the 3′ end of the strand only). The other nucleotides of the first strand are linked by phosphodiester linkages.

Sequences are shown in the conventional 5′ to 3′ direction.

The first and second strands are described by SEQ ID NOs: 165 and 164 respectively of WO 2020/099476, which describes SLN360 as “conjugate 21”. The ligand moiety [M], (including its phosphorothioate link to the second strand) is designated in that document as “[ST23 (ps)]3 C6XLT (ps)”.

### Conditions

Conditions associated with elevated levels of Lp(a) lipoprotein, e.g., with elevated levels of Lp(a)-containing particles, include cardiovascular diseases and some cancer (including prostate cancer).

Thus, the condition may, for example, be a cardiovascular disease (e.g., an atherosclerotic cardiovascular disease or atherosclerotic cerebrovascular disease) such as stroke (e.g., ischaemic stroke), atherosclerosis, thrombosis, coronary heart disease, aortic stenosis (e.g., calcific aortic stenosis), hyperlipidaemia, dyslipidaemia, coronary artery disease, peripheral arterial disease, abdominal aortic aneurysm, heart failure (e.g., heart failure secondary to ischaemic cardiomyopathy), or familial hypercholesterolaemia, all preferably when associated with elevated levels of Lp(a).

Alternatively, the condition may be a cancer, e.g., prostate cancer.

The condition is associated with elevated Lp(a) (i.e., lipoprotein A) in a subject, e.g., with elevated levels of Lp(a)-containing particles in a subject. The subject may be an adult (18 years of age or above) or a child (under 18 years of age).

It will be understood that the relevant level of Lp(a) or Lp(a)-containing particles is typically measured in the subject's serum. A desirable level of Lp(a)-containing particles in serum is generally described as a level of under 14 mg/dL. An elevated level of Lp(a)-containing particles is a level of at least 14, preferably at least 20, more preferably at least 30, more preferably at least 40 and most preferably at least 50 mg/dL or at least 60 mg/dL of Lp(a)-containing particles in the serum of a subject.

The terms “Lp(a)-containing particles” and “Lp(a) particles” may be used interchangeably.

### Pharmaceutical Compositions

SLN360 may be formulated for administration, e.g., as part of a pharmaceutical composition, alone or in combination with one or more other agents. The pharmaceutical composition may comprise a pharmaceutically acceptable excipient, such as a stabilizer, preservative, diluent, buffer, or the like. For example, the SLN360 molecule may be combined with a delivery vehicle (e.g., liposomes) and/or excipients, such as carriers or diluents. Other agents such as preservatives and stabilizers can also be added. Methods for the delivery of nucleic acids are known in the art and within the knowledge of the person skilled in the art.

The pharmaceutical composition may be a sterile injectable aqueous suspension or solution, or in a lyophilised form or adhered, absorbed or included to or into any other suitable galenic carrier substance such as pellets, tablets, capsules, nanoparticles, gels, tablets, beads or similar structures.

However, a simple aqueous solution may be adequate, e.g., at a physiologically acceptable pH value of 6.4-8.4. It may be desirable that the composition has a pH of 7.6 +/−0.1.

The SLN360, or the pharmaceutical composition, may be administered (or formulated for administration) by any appropriate route, e.g., via subcutaneous, intravenous, oral, rectal or intraperitoneal administration. It may be administered (or formulated for administration) by injection. Preferably administration is by subcutaneous injection, e.g., delivered into the abdomen, upper arm or thigh.

The invention further provides an individual dosage form of SLN360, providing an individual dose of SLN360 as described herein. Preferably the individual dosage form is for administration by subcutaneous injection. It may be an aqueous solution as described, e.g., having a pH of 6.4-8.4, e.g., having a pH of 7.6+/−0.1.

The terms “patient,” “subject,” and “individual” may be used interchangeably and refer to either a human or a non-human animal. These terms include mammals such as humans, non-human primates (e.g., great ape, old world monkey or new world monkey), livestock animals (e.g., bovines, porcines), companion animals (e.g., canines, felines) and rodents (e.g., mice and rats). However, in the context of the present invention, the subject is typically human. The subject may be an adult (18 years of age or above) or a child (under 18 years of age). Thus, the terms LPA, LPA, Lp(a), “Lp(a)-containing particles” and “Lp(a) particles” should typically be taken to refer to human proteins (or lipoproteins) and nucleic acids, unless the context demands otherwise.

As used herein, “treating” or “treatment” and grammatical variants thereof refer to an approach for obtaining beneficial or desired clinical results. The term may refer to slowing the onset or rate of development of a condition, disorder or disease, reducing or alleviating symptoms associated with it, generating a complete or partial regression of the condition, or some combination of any of the above. For the purposes of this invention, beneficial or desired clinical results include, but are not limited to, reduction or alleviation of symptoms, diminishment of extent of disease, stabilization (i.e., not worsening) of state of disease, delay or slowing of disease progression, amelioration or palliation of the disease state, and remission (whether partial or total), whether detectable or undetectable. “Treatment” can also mean prolonging survival relative to expected survival time if not receiving treatment. A subject (e.g., a human) in need of treatment may thus be a subject already afflicted with the disease or disorder in question. The term “treatment” includes inhibition or reduction of an increase in severity of a pathological state or symptoms relative to the absence of treatment, and is not necessarily meant to imply complete cessation of the relevant disease, disorder or condition.

As used herein, the terms “prophylaxis” and grammatical variants thereof refer to an approach for inhibiting or preventing the development, progression, or time or rate of onset of a condition, disease or disorder, and may relate to pathology and/or symptoms. For the purposes of this invention, beneficial or desired clinical results include, but are not limited to, prevention, inhibition or slowing of symptoms, progression or development of a disease, whether detectable or undetectable. A subject (e.g., a human) in need of prophylaxis may thus be a subject not yet afflicted with the disease or disorder in question. The term “prophylaxis” includes slowing the onset of disease relative to the absence of treatment and is not necessarily meant to imply permanent prevention of the relevant disease, disorder or condition. Thus “prophylaxis” of a condition may in certain contexts refer to reducing the risk of developing the condition, or preventing, inhibiting or delaying the development of symptoms associated with the condition. It will be understood that prophylaxis may be considered as treatment or therapy.

The pharmaceutical compositions and medicaments of the present invention may be administered to a subject in a therapeutically or prophylactically effective amount, i.e., an effective dose. As used herein, an “effective amount,” “prophylactically effective amount”, “therapeutically effective amount” or “effective dose” is an amount of a composition (e.g., a therapeutic composition or agent) that produces at least one desired therapeutic effect in a subject, such as preventing or treating a target condition or beneficially alleviating a symptom associated with the condition.

As used herein, the term “pharmaceutically acceptable salt” refers to a salt that is not harmful to a patient or subject to which the salt in question is administered. It may be a salt chosen, e.g., among acid addition salts and basic salts. Examples of acid addition salts include chloride salts, citrate salts and acetate salts. Examples of basic salts include salts wherein the cation is selected from alkali metal cations, such as sodium or potassium ions, alkaline earth metal cations, such as calcium or magnesium ions, as well as substituted ammonium ions, such as ions of the type N(R¹)(R²)(R³)(R⁴)+, wherein R¹, R², R³ and R⁴ independently will typically designate hydrogen, optionally substituted C1-6-alkyl groups or optionally substituted C2-6-alkenyl groups. Examples of relevant C1-6-alkyl groups include methyl, ethyl, 1-propyl and 2-propyl groups. Examples of C2-6-alkenyl groups of possible relevance include ethenyl, 1-propenyl and 2-propenyl. Other examples of pharmaceutically acceptable salts are described in “Remington's Pharmaceutical Sciences”, 17th edition, Alfonso R. Gennaro (Ed.), Mark Publishing Company, Easton, PA, USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66: 2 (1977). A “pharmaceutically acceptable salt” retains qualitatively a desired biological activity of the parent compound without imparting any undesired effects relative to the compound. Examples of pharmaceutically acceptable salts include acid addition salts and base addition salts. Acid addition salts include salts derived from nontoxic inorganic acids, such as hydrochloric, nitric, phosphorous, phosphoric, sulfuric, hydrobromic, hydroiodic and the like, or from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include salts derived from alkaline earth metals, such as sodium, potassium, magnesium, calcium and the like, as well as from nontoxic organic amines, such as N, N′-dibenzylethylenediamine, N-methylglucamine, chloroprocaine, choline, diethanolamine, ethylenediamine, procaine and the like.

The term “pharmaceutically acceptable carrier” includes any of the standard pharmaceutical carriers. Pharmaceutically acceptable carriers for therapeutic use are well known in the pharmaceutical art, and are described, for example, in Remington's Pharmaceutical Sciences, Mack Publishing Co. (A. R. Gennaro edit. 1985). For example, sterile saline and phosphate-buffered saline at slightly acidic or physiological pH may be used. Exemplary pH buffering agents include phosphate, citrate, acetate, tris/hydroxymethyl)aminomethane (TRIS), N-Tris(hydroxymethyl)methyl-3-aminopropanesulphonic acid (TAPS), ammonium bicarbonate, diethanolamine, histidine, which is a preferred buffer, arginine, lysine, or acetate or mixtures thereof. The term further encompasses any agents listed in the US Pharmacopeia for use in animals, including humans. A “pharmaceutically acceptable carrier” includes any and all physiologically acceptable, i.e., compatible, solvents, dispersion media, coatings, antimicrobial agents, isotonic and absorption delaying agents, and the like. In certain embodiments, the carrier is suitable for intravenous, intramuscular, subcutaneous, parenteral, spinal or epidermal administration (e.g., by injection or infusion). Depending on selected route of administration, the nucleic acid may be coated in a material or materials intended to protect the compound from the action of acids and other natural inactivating conditions to which the nucleic acid may be exposed when administered to a subject by a particular route of administration.

The term “solvate” in the context of the present invention refers to a complex of defined stoichiometry formed between a solute (in casu, a nucleic acid compound or pharmaceutically acceptable salt thereof according to the invention) and a solvent. The solvent in this connection may, for example, be water or another pharmaceutically acceptable, typically small-molecular organic species, such as, but not limited to, acetic acid or lactic acid. When the solvent in question is water, such a solvate is normally referred to as a hydrate.

The invention will now be described with reference to the following non-limiting Tables, Figures and Examples.

### FIGURES AND TABLES

FIG. 1. Flow of patients through the trial.

FIGS. 2A-2B. Panel A shows the changes over time in lipoprotein(a) concentration for the placebo and each SLN360 dose group. The median lipoprotein(a) concentration is shown as solid circles and the boxes show the interquartile ranges for each of these values. The stars show the mean concentration at each time point. Panel B. shows percent changes in lipoprotein(a) concentration over time for the placebo and each SLN360 dose group. The median percent change in lipoprotein(a) concentration are shown as solid circles and the boxes show the interquartile ranges for each of these values. The stars show the mean percent change in concentration at each time point.

FIGS. 3A-3B. Panel A shows mean percent changes in low-density-lipoprotein-cholesterol over time for the placebo and each SLN360 dose group. The mean percent changes are shown as solid circles with the 95% confidence intervals shown as error bars. Panel B shows mean percent changes in apolipoprotein B over time for the placebo and each SLN360 dose group. The mean percent changes are shown as solid circles with the 95% confidence intervals shown as error bars.

FIG. 4. Study schematic. Cohort 5: two doses, 200 mg q4 weeks. Cohort 6: two doses 300 mg q8 weeks.

FIG. 5. Time averaged Results Over 0-150 days.

FIG. 6. Time averaged Results Over 30-150 days.

Table 1. Baseline Characteristics of Participants

Table 2. Safety Assessments

Table 3. Primary Efficacy Outcome: Change in Lipoprotein (a) Concentration over Time

Table 4. Effect of SLN360 on Total Cholesterol

Table 5. Effect of SLN360 on Low-Density Lipoprotein Cholesterol

Table 6. Effect of SLN360 on Apolipoprotein(B)

Table 7. Effect of SLN360 on Oxidized LDL

Table 8. Pharmacokinetic Parameters

### EXAMPLES

### Methods

### Study Population

Adults 18 years in age with no known atherosclerotic cardiovascular disease (ASCVD), an Lp(a) concentration of 150 nmol/L and a body mass index of 18 to 45 kg/m² were eligible to participate. Women of child-bearing potential were required to have a negative serum pregnancy test at screening and a negative urine pregnancy test 1 day prior to treatment. All participants (male and female) agreed to adhere to contraception requirements from the beginning of the screening period until 3 months after the last administration of study drug. Participants were excluded for moderate or severe hepatic cirrhosis, a positive test for HIV or hepatitis B or C, or other liver disease that could increase the risk of drug-induced liver injury or influence the pharmacology of SLN360. Participants were required to have an HbA1c<6.5% if not diagnosed previously with diabetes or <8.5% if known to have diabetes.

Laboratory exclusion criteria included a platelet count below the lower limit of normal or an alanine transaminase or aspartate aminotransferase >1.5× upper limit of normal (ULN). Additional laboratory exclusion criteria were total bilirubin >ULN, except in participants with Gilbert's syndrome, or an estimated glomerular filtration rate <60 mL/min/1.73 m².

Medications that could influence Lp(a) levels required doses to be stable for at least 8 weeks prior to enrollment, including anti-estrogen or estrogen receptor modulators, PCSK9 inhibitors, prescription dose niacin, fibrates, statins or ezetimibe. Participants were also excluded if they had received any other investigational agent within 90 days (or 10 half-lives, whichever was longer) or other oligonucleotide therapy within 12 months before the first dose of study drug. Participants were excluded for clinically significant illness within 7 days before the first dose of study drug, a positive nucleic acid test for SARS-CoV-2, or any uncontrolled or serious disease that could interfere with the interpretation of results.

### Study Procedures

This study was conducted in accordance with the Declaration of Helsinki and Council for International Organizations of Medical Sciences International Ethical Guidelines. Four ascending single dose cohorts were randomized using an interactive response technology to 30 mg, 100 mg, 300 mg or 600 mg of SLN360 or placebo (sodium chloride 0.9%) given by subcutaneous injection in the abdomen. Up to two injection sites were used to administer the required dose. For each cohort, 2 participants were randomly assigned to receive placebo and 6 subjects assigned to SLN360 with sentinel dosing employed for the first two participants in each cohort. A schematic describing the study design is provided in FIG. 4. The investigators, study participants and the sponsor were blinded to study drug, but delegated site staff e.g., the site pharmacist were unblinded. Participants were monitored in a clinical research unit (CRU) for 24 hours following dose administration during which vital signs, an abbreviated physical examination, blood and urine samples and serial electrocardiograms were obtained. CRU staff also assessed participants for adverse effects and injection site adverse events.

Participants had scheduled visits at day 7, 14, 30, 45, 60, 90 and 150 following drug administration. The presence and extent of any adverse effects of study drug administration were assessed at each visit. At all return visits, blood samples were obtained for lipoprotein(a) levels, other biomarkers, pharmacokinetic analyses and safety laboratory studies, and, if indicated by symptoms, a focused physical examination was performed. Lp(a) concentration was measured with a particle-enhanced turbidimetric assay on a Roche c502 autoanalyser. This assay is calibrated by five independent standards with values in nmol/L traceable to the WHO/IFCC Reference Material for Lp(a) and has been demonstrated to be minimally affected by the particle size variation of apo(a).

### Study Endpoints

As a first-in-human study, the safety and tolerability of SLN360 were defined as a primary outcome of interest. Safety assessments included changes in vital signs, physical examination, ECG and laboratory assessments of chemistry, hematology, urinalysis and coagulation parameters. Assessments included treatment emergent adverse events (TEAE) and adverse events of special interest, which included injection site adverse events or any identified dose-limiting toxicity. Injection site adverse events were graded by study personnel using both the Common Terminology Criteria for Adverse Events (CTCAE)⁹ version 5 and an FDA-endorsed scale¹⁰.

The primary efficacy assessment was the effect of SLN360 on plasma lipoprotein(a) concentrations from baseline to 150 days following dosing. Secondary objectives included assessment of the pharmacokinetics (PK) of SLN360. Exploratory objectives included assessment of effects on other lipid parameters (LDL-C, HDL-C, total cholesterol, and triglycerides), apolipoprotein B, oxidized LDL, inflammatory markers, and plasminogen.

### Statistical Analysis

Subjects assigned to placebo from each cohort were pooled to create an overall placebo treatment group. SLN360 treatment groups are presented separately by dose. Descriptive statistics are used to summarize safety and Lp(a) change by treatment group. For categorical variables, summary tabulations of frequency and percentage of subjects within each category are presented. For continuous variables, the number of participants and summary statistics are presented by treatment group. For pharmacokinetic effects, the plasma SLN360 concentration includes maximum plasma concentration (Cₘₐₓ)), time to reach peak plasma concentration (Tₘₐₓ) and area-under-the-concentration curve to last measurable concentration (AUC₍₀₋ₗₐₛₜ₎). Missing data were not imputed. No formal statistical testing was conducted and therefore no P values are reported. The protocol prespecified that the effects of single ascending doses of SLN360 would be reported separately from the subsequent multiple ascending dose phase of the trial. This report describes the findings of the single ascending dose part.

### Results

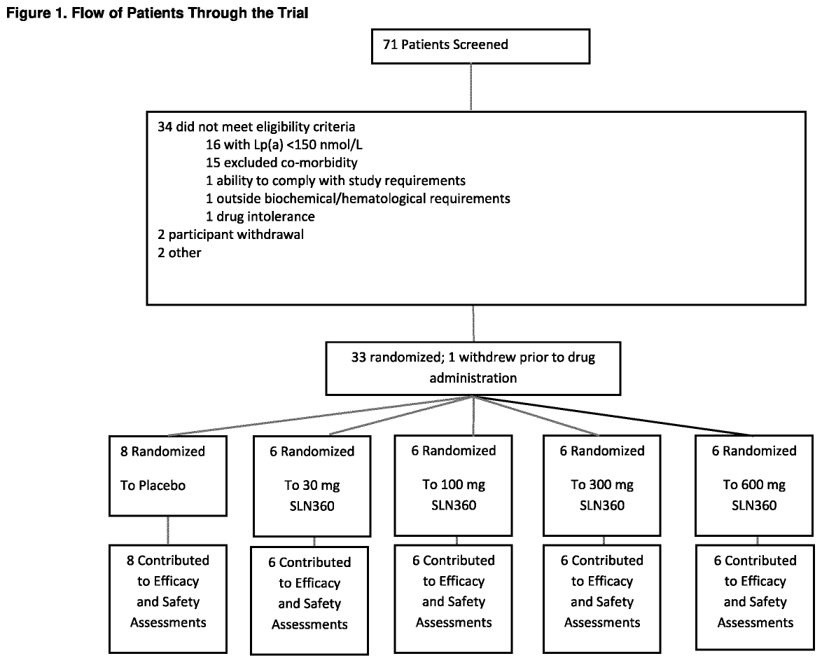

FIG. 1 shows the flow of patients through the trial. Table 1 reports the baseline characteristics of the trial participants. The mean (SD) age of participants was 50 years (13.5) and 47% were male. Although none of the participants had known pre-existing clinically overt cardiovascular disease, hypertension was present in 9% and diabetes in 3%. Baseline characteristics appeared broadly similar across all treatment groups. Median (IQR) baseline Lp(a) concentrations were 238 (203, 308), 171 (142, 219), 217 (202, 274), 285 (195, 338) and 231 (179, 276) nmol/L for the placebo, 30 mg, 100 mg, 300 mg, and 600 mg treatment groups, respectively. Mean (SD) levels of LDL-C were 99 (48), 113 (38), 121 (46), 100 (25) and 108 (54) mg/dL for the placebo, 30 mg, 100 mg, 300 mg, and 600 mg treatment groups, respectively.

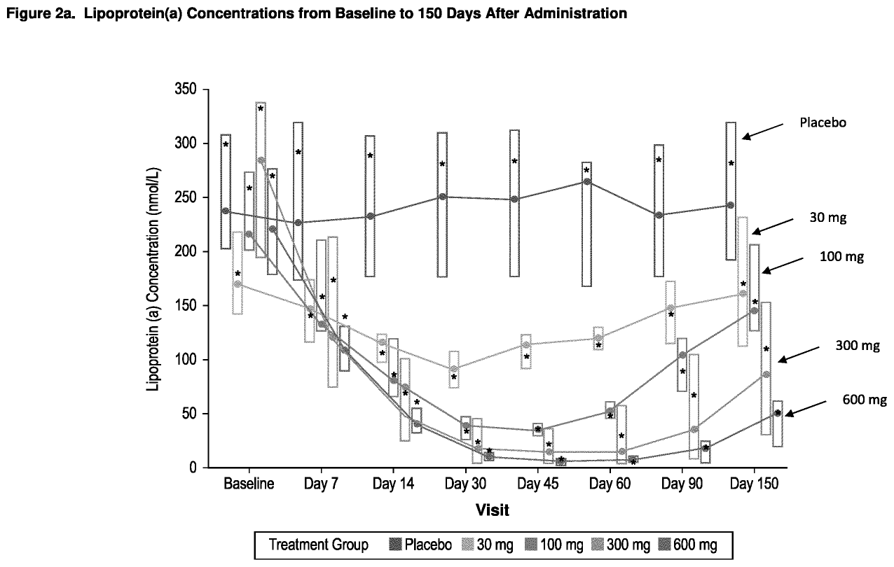

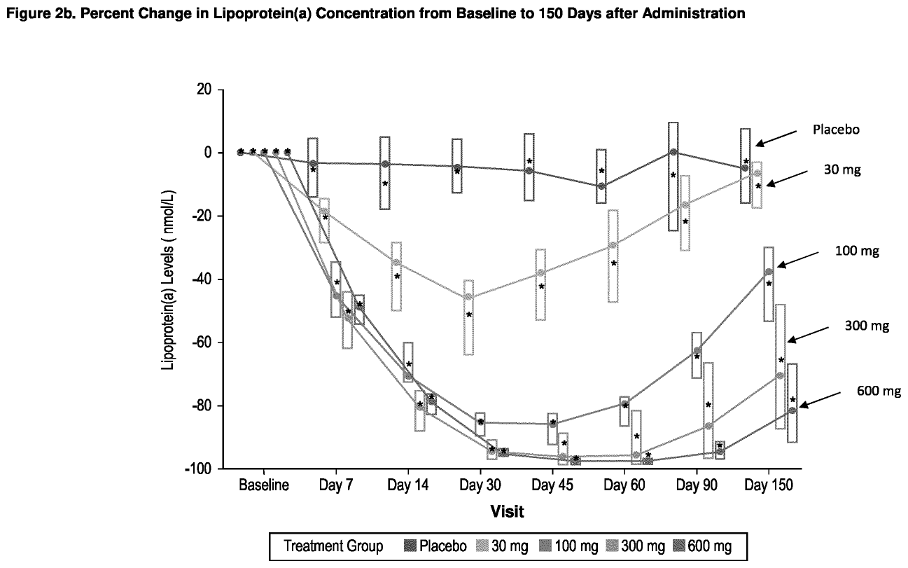

FIGS. 2a and 2b show the absolute and percent changes over time in Lp(a) concentration for each treatment group for 150 days following injection. Table 3 shows the median values and IQRs for absolute and percent changes at each time point. The maximal median (IQR) changes in Lp(a) in nmol/L were −20 (−61, 3), −89 (−119, −61), −185 (−226, −163), −268 (−292, −189) and −227 (−270, −174) for the placebo, 30 mg, 100 mg, 300 mg, and 600 mg doses, respectively. The maximal median (IQR) percent changes were −10% (−16, 1%), −46% (−64, −40%), −86% (−92, −82%), −96% (−98, −89%) and −98% (−98, −97%) for the placebo, 30 mg, 100 mg, 300 mg, and 600 mg groups, respectively. The nadir of percent change in Lp(a) levels was achieved at between 30 and 60 days after dosing for all treatment groups. (FIG. 2). Concentrations gradually rose from the nadir, but had not returned to baseline values by 150 days for the 100 mg, 300 mg, and 600 mg treatment groups. Median Lp(a) concentrations were >70% and >80% below baseline 150 days following administration for the 300 mg and 600 mg doses, respectively.

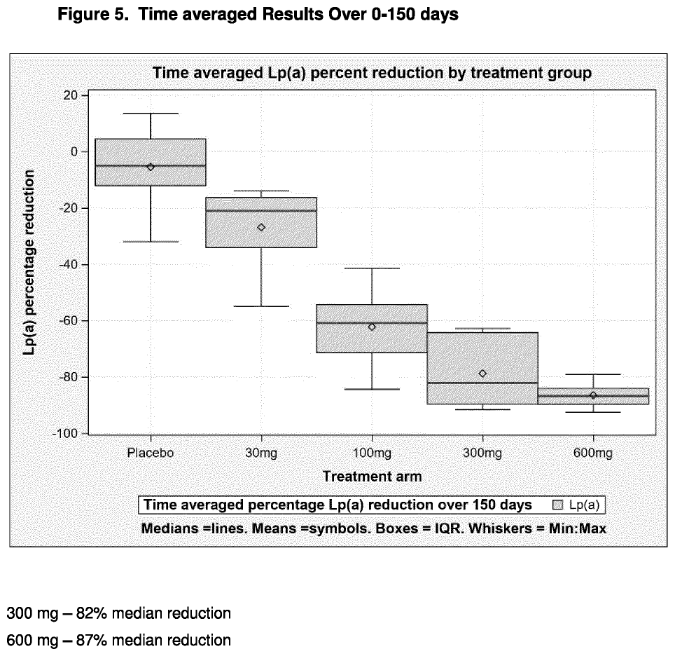

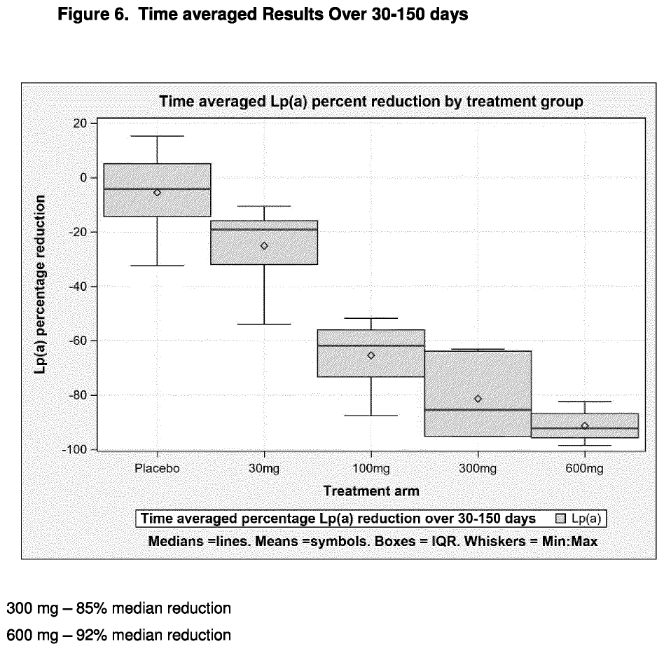

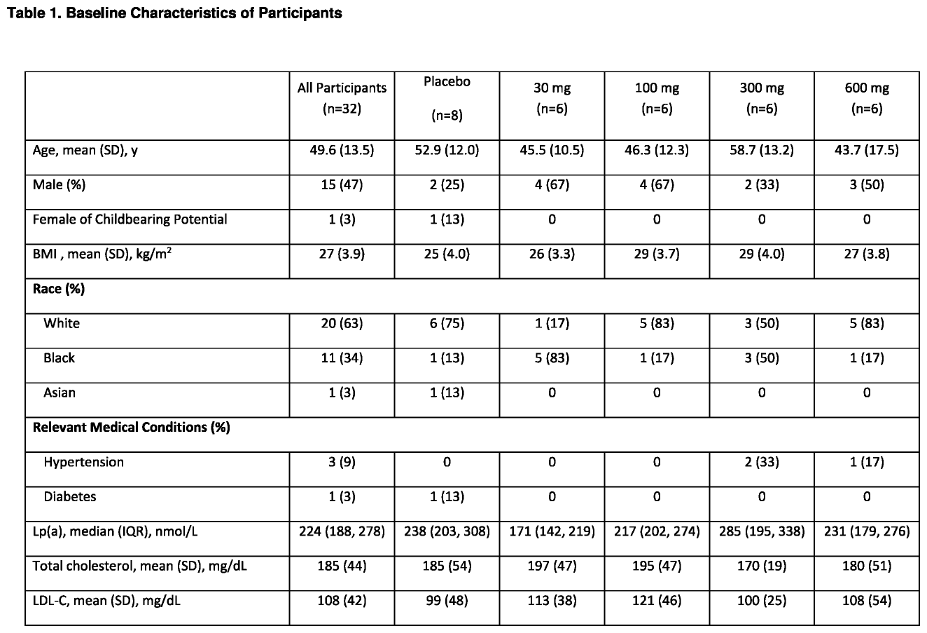

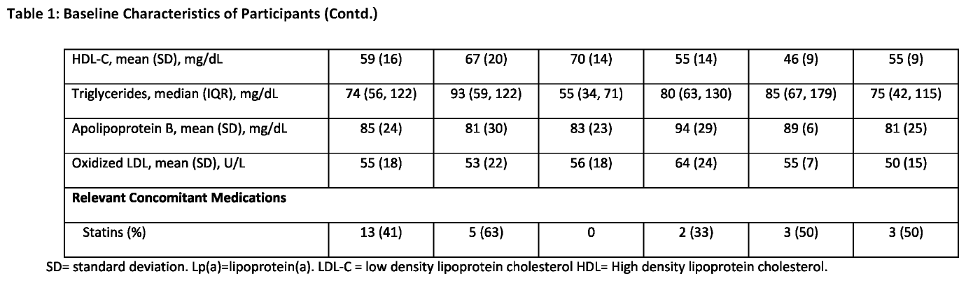

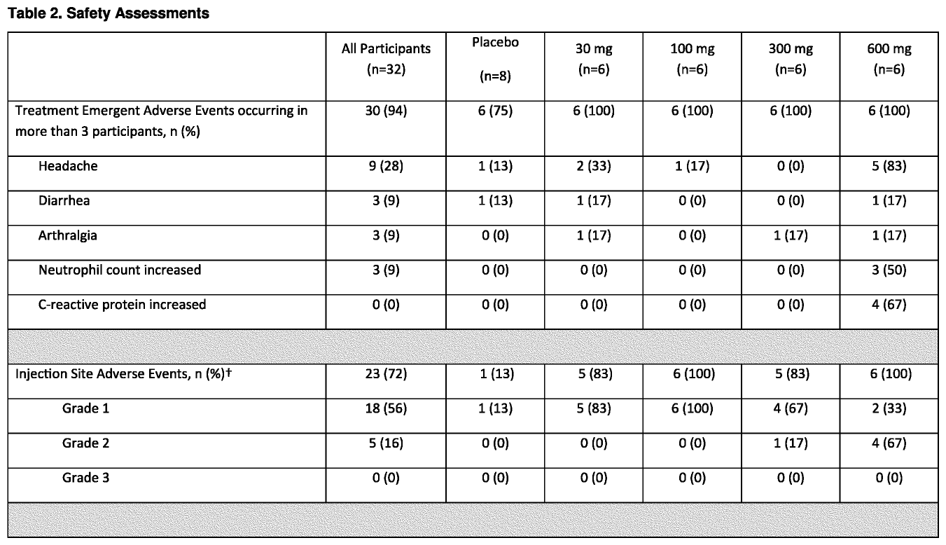

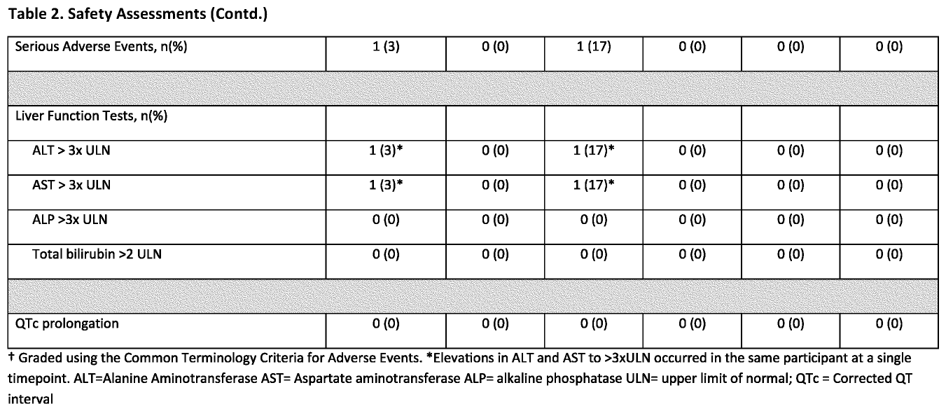

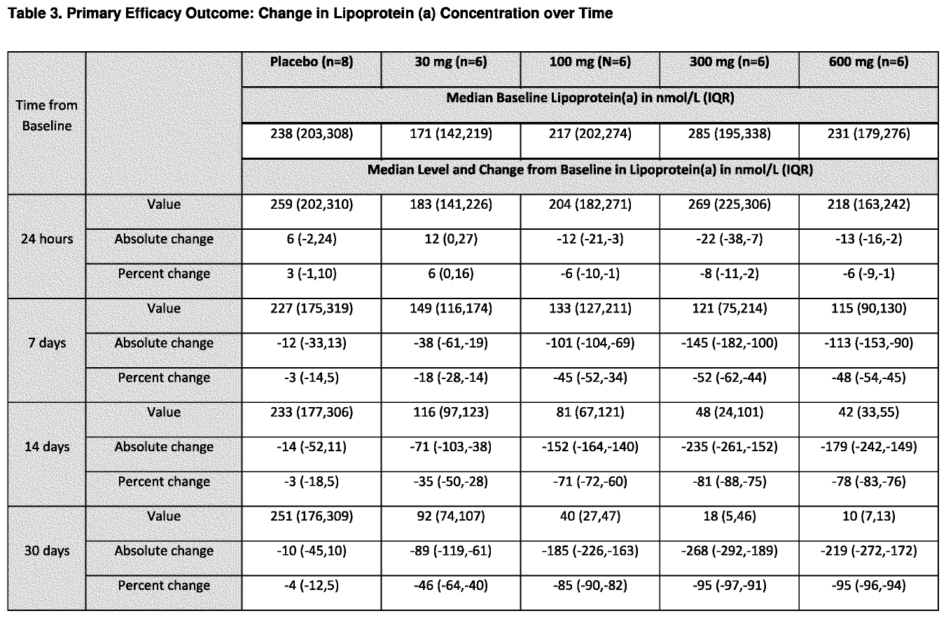

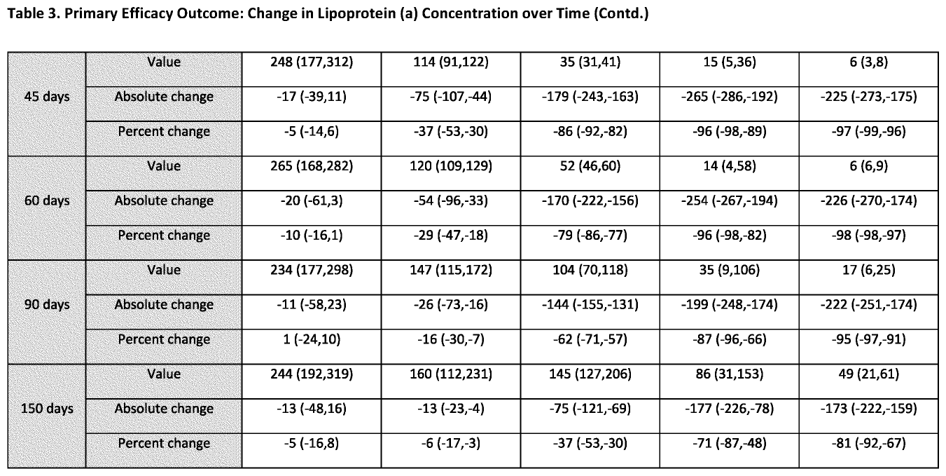

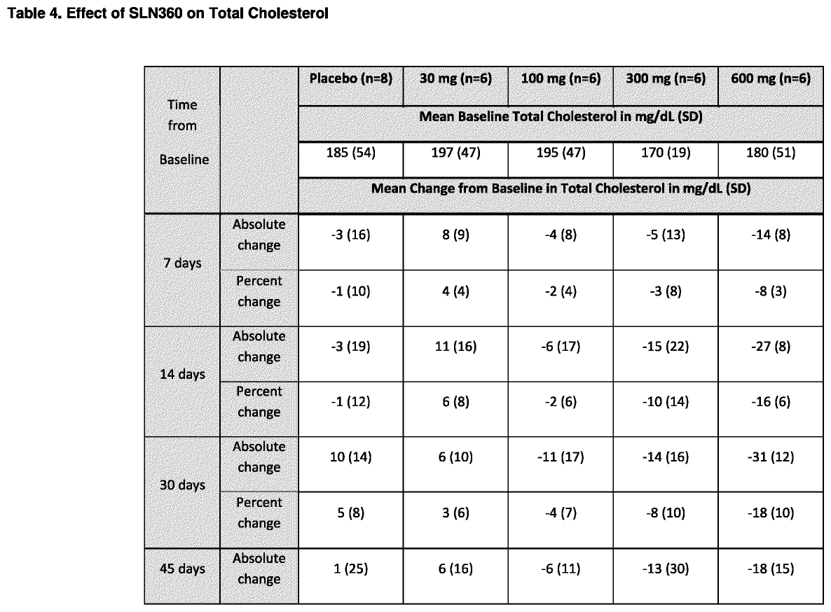

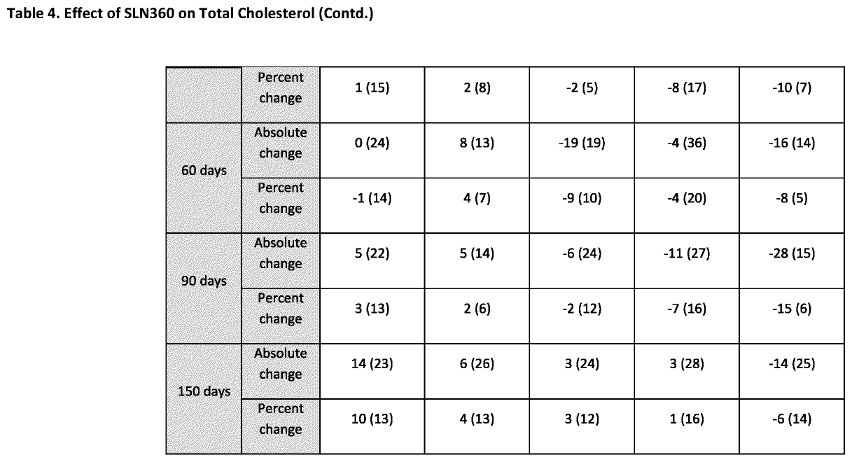

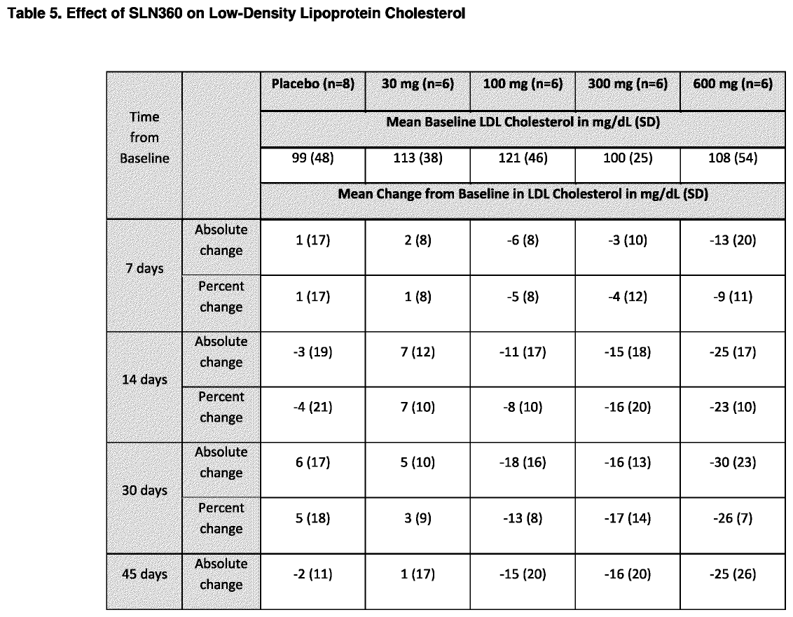

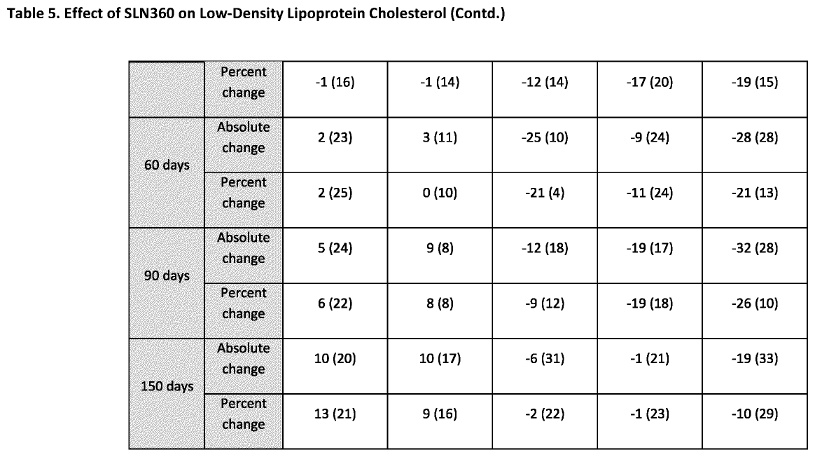

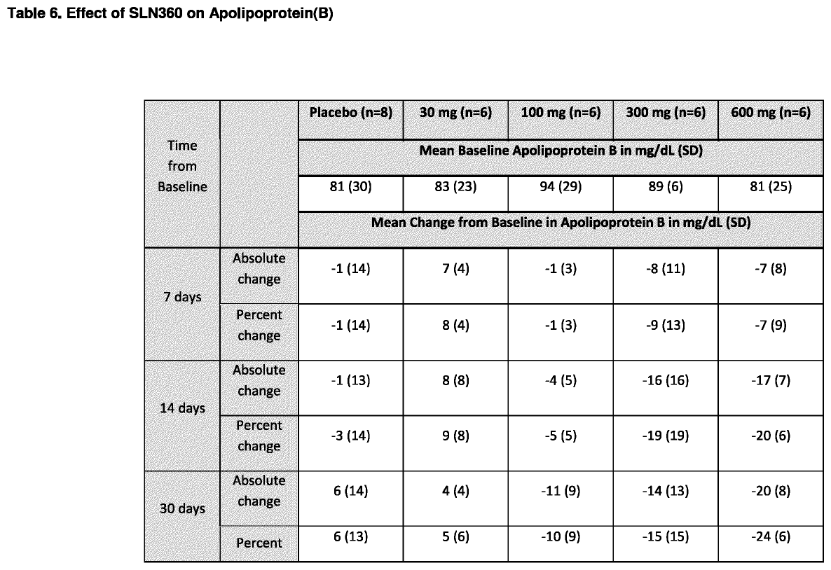

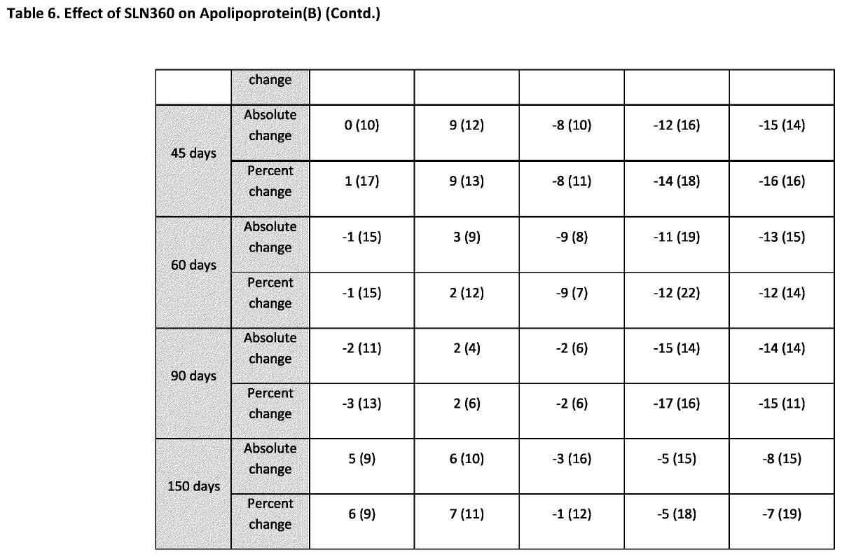

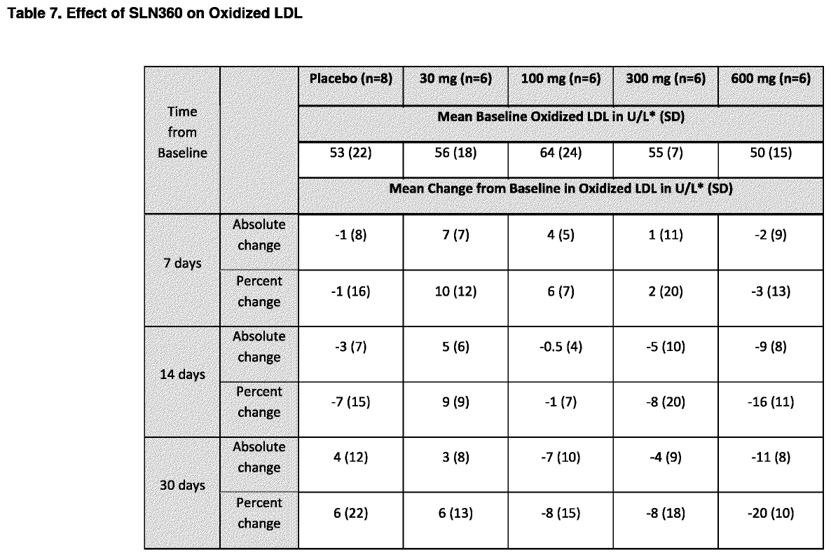

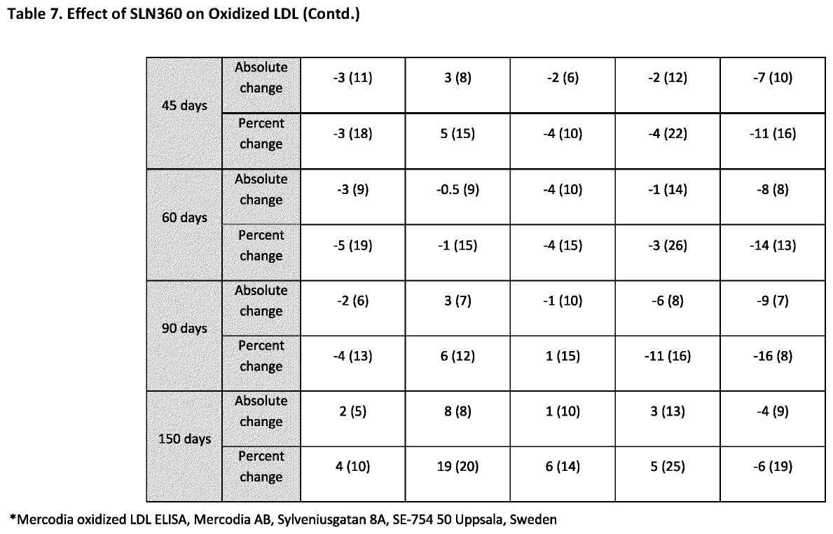

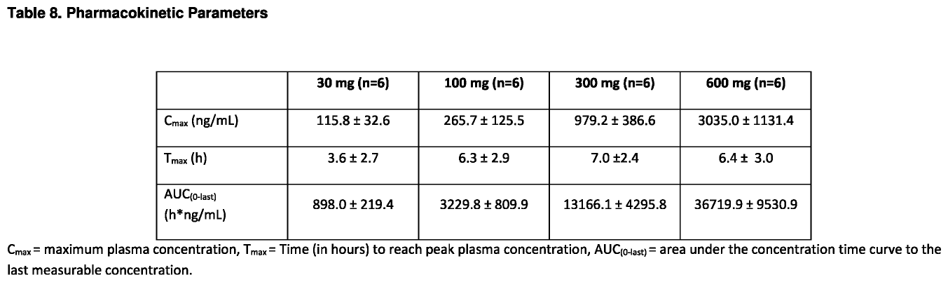

Time-averaged reductions were determined to further inform dosing frequency and better evaluate potential clinical benefits. FIGS. 5 and 6, respectively, show time-averaged data including the total observation period up to day 150 (a conservative estimate as it includes the original baseline prior to any treatment effect); and the observation period of days 30-150 (more reflective of the drug effect after the nadir has been achieved by original dosing.) Time averages are considered useful in evaluating the cumulative effect of multiple dosing, capturing peaks and troughs of drug effect.

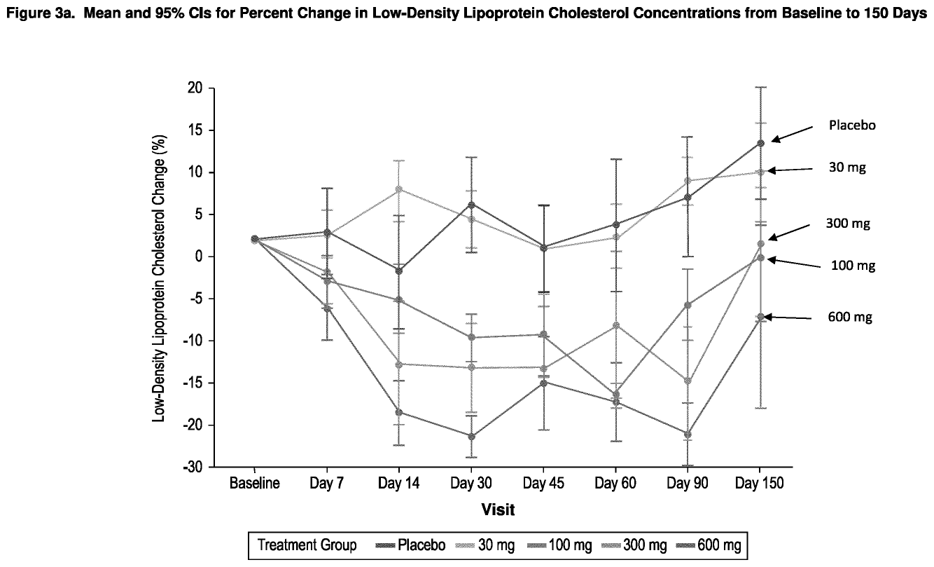

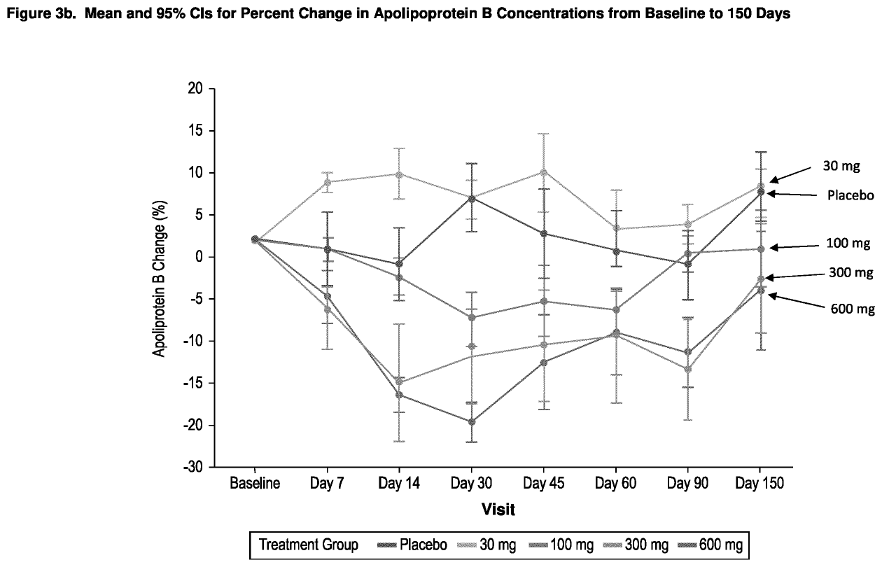

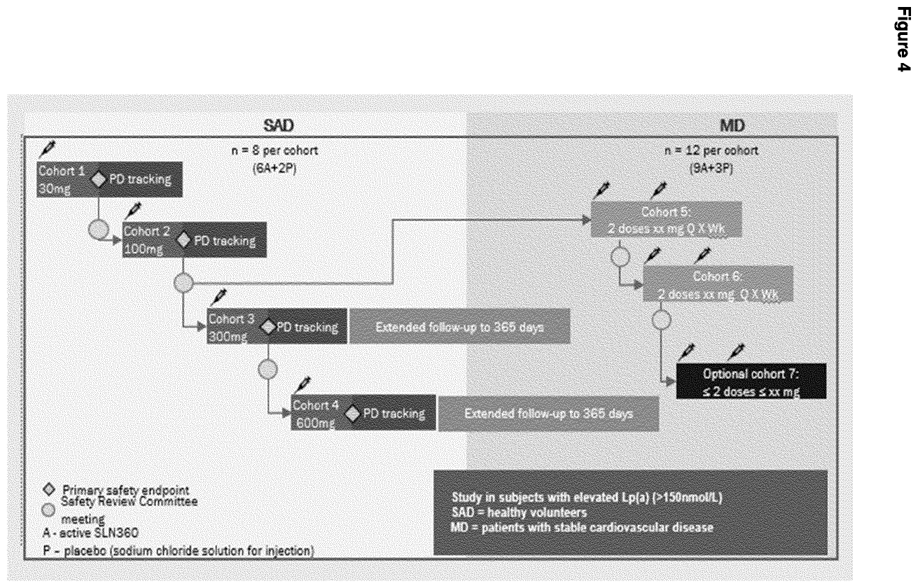

SLN360 produced a moderate, dose-dependent reduction in apolipoprotein (B) containing lipoproteins (Tables 4-7). Mean levels were reduced by a maximum of 18% for total cholesterol and 26% for LDL-C, both following administration of the 600 mg dose. (FIG. 3a and Tables 4,5) Lesser reductions were observed at lower doses. Maximum reduction in the mean level of apolipoprotein B was 24% measured 30 days after the 600 mg dose and 19% measured 14 days after the 300 mg dose. (FIG. 3b and Table 6) Mean levels of oxidized LDL were lowered by a maximum of 20% in the 600 mg dose group and 11% in the 300 mg dose group with similar reductions sustained out to 150 days in the 600 mg group. (Table 7) There was no evidence of an effect of SLN360 on triglycerides and HDL-C. Table 8 shows that plasma pharmacokinetics of SLN360 were broadly dose proportional.

Table 2 reports safety findings for each treatment group including assessment of injection site adverse events graded by the CTCAE scale.¹⁰

TEAEs were generally mild, most commonly low-grade injection site events (grades 1 and 2) and headache with none resulting in participant withdrawal. (Table 2) At day 45, after receiving a SARS-CoV-2 vaccine on day 38, a single participant in the lowest dose group (30 mg) was admitted to the hospital for fever and a severe headache, which were ascribed by the investigator to effects of the SARS-CoV-2 vaccine. The subsequent planned day 45 visit was conducted on day 49 and the participant was found to have a >3×ULN value for alanine aminotransferase (ALT) and aspartate aminotransferase (AST) with no elevation in bilirubin. The AST and ALT had returned to normal at the routine day 60 laboratory testing.

Transient, dose-dependent increases in C-reactive protein (CRP) occurred within the first 24 hours after administration. In the 600 mg dose group, CRP rose from a median of 1.0 mg/L to 16.3 mg/L at 24 hours, then rapidly decreased reaching 1.1 mg/L by day 14. (not shown).

Neutrophils also increased during the first 24 hours, returning to normal by the day 7 visit.

Following data review, the SRC recommended extending follow-up of Lp(a) levels for participants in the highest two dose groups from 150 days to 365 days (1 year). These extension studies included 3 placebo subjects, 5 subjects in cohort 3 (300 mg; 4 subjects completed) and 6 subjects in cohort 4 (600 mg). At day 365, some subjects in the 300 mg and 600 mg cohorts still exhibited substantial knockdown of Lp(a) up to 50% of baseline whereas subjects in the placebo arm had all returned to baseline at day 365. No new safety findings and no serious adverse effects related to study drug were observed in either of the highest two dose groups at 365 days.

### Discussion

Patients with Lp(a) concentrations greater than 150 nmol/L (approximately 60 mg/dL) comprise approximately 20% of the population and are associated with greatly increased risk for developing ASCVD and aortic valve stenosis.⁴⁻⁵ The APOLLO trial enrolled participants with plasma Lp(a) concentrations exceeding this threshold. Plasma Lp(a) levels are genetically determined and are not influenced by lifestyle interventions, such as diet and exercise, that lower LDL-C and triglycerides or raise HDL-C. In the absence of an effective Lp(a)-lowering drug therapy, this risk factor has traditionally been considered essentially untreatable. Recent developments using RNA interference have made it possible to selectively and substantially reduce Lp(a). The first of these drugs to reach phase 3, an anti-sense oligonucleotide (ASO), pelacarsen, reduced Lp(a) by up to 80% with weekly injections in a phase 2 trial with subsequent development as a monthly therapy.⁸

The current trial examined an alternative approach to preventing the hepatic assembly of Lp(a) particles, using an siRNA (SLN360) that degrades the messenger RNA that encodes for production of apolipoprotein(a).¹¹ Although SLN360 is still in early development, the preliminary results of the current phase 1 study are encouraging, with median Lp(a) concentrations decreasing up to 98% with persistent median reductions exceeding 70% and 80% at 150 days following administration of the 300 mg and 600 mg doses, respectively. (Table 3 and FIG. 2) Following data review, the SRC recommended extending follow-up of Lp(a) levels for participants in the highest two dose groups (cohrots 3 and 4) from 150 days to a year. No new safety findings and no serious adverse effects related to study drug were observed in subjects of these highest two dose groups at 365 days and there were still signs of substantial knockdown of Lp(a) in some subjects of each cohort compared to subjects in the placebo group. In Phase 1 studies, evaluation of safety and tolerability is the principal objective. The current study was small, including only 32 participants without known cardiovascular disease studied for 150 days after a single administration of SLN360. Since this therapy targets Lp(a) production in hepatocytes, the study measured several markers of liver function to monitor for hepatotoxicity. During the study, one episode of a transient 3-fold elevation in liver enzymes (AST and ALT) was observed in a single patient who received the lowest dose (30 mg), temporally associated with SARS-CoV-2 vaccination. Elevation of liver enzymes has been previously described after COVID vaccination.¹² No other clinically important derangements in liver function were observed. Definitive conclusions regarding hepatic safety are not possible since only a small number of participants were treated for a short period of time.

Similar to other RNAi therapies, low-grade injection site adverse events were observed (Table 2). These adverse events were self-limiting with no lasting effects and did not lead to participant withdrawal. Transient, dose-dependent increases in inflammation biomarkers, including CRP and neutrophils, were observed in the first 7 days after administration. The clinical significance of these short-term increases is uncertain. During the initial development of some prior ASO therapies, thrombocytopenia was observed at higher weekly dosing, but we did not observe any clinically relevant platelet reductions in the current study with this GalNAc siRNA.¹³ Future studies treating patients with multiple doses for a longer exposure time will be required to more definitively establish the safety of this siRNA therapeutic agent.

The current study also examined the effects of SLN360 on a range of lipids and lipoproteins, including LDL-C, apoB, triglycerides, HDL-C and oxidized LDL. Both LDL-C and apoB were moderately reduced, a maximum of approximately 25%, most evident at the highest doses. (FIGS. 3a and 3b, Tables 5 and 6) A moderate reduction in LDL-C and apoB was expected since current assays also measure a portion of the LDL-C and apoB contained within Lp(a) particles.¹⁴ Hence, as Lp(a) is lowered, the measured LDL-C and apoB concentrations also fall. Oxidized LDL is present in Lp(a) particles and are pro-inflammatory, which may contribute to the risk of atherothrombotic events.¹⁵ Mean values for oxidized LDL were lowered by a maximum of 20% and remained substantially lower than baseline for 45-60 days in the lower dose groups and 150 days for the 600 mg dose group. (Table 7)

Inhibiting translation through RNA interference is increasingly being used in therapeutic drug development to inhibit hepatic production of specific target proteins.¹¹ An siRNA is a double-stranded RNA with guide (antisense) and passenger (sense) strands designed to specifically suppress the translation of a target gene with mRNA containing a sequence complementary to the guide strand. Once the siRNA enters the cell, it is incorporated into an RNA-induced silencing complex (RISC). The guide strand and passenger strand are separated, the passenger strand is cleaved and degraded, and the remaining single-stranded siRNA guide strand binds to the target mRNA, inducing cleavage through a catalytic mechanism leaving the guide strand intact and available to degrade additional copies of the target mRNA. The cleaved mRNA leaves RISC and is recognized as abnormal and degraded by cellular exonucleases. This process prevents translation of this specific mRNA into protein. In this fashion, siRNA therapeutics can have a very long duration of effect. A recently published phase 1 study of olpasiran, a different siRNA targeting Lp(a) also showed large, dose-dependent and durable lowering of Lp(a).¹⁶ The siRNA used in this trial was conjugated with GalNAc, a sugar derived from galactose, enabling targeted delivery of SLN360 to the liver. This approach is now used in most hepatically-directed RNA interference therapeutics currently marketed or under development. GalNAc binds to asialoglycoprotein receptors which are highly expressed on the surface of hepatocytes leading to rapid endocytosis of the conjugate. After entering hepatocytes, the acidification during endosomal maturation leads to dissociation of the GalNAc ligand that delivered the siRNA to the cell. The selective update of SLN360 in hepatocytes reduces the administered dose of this siRNA needed to degrade LPA mRNA which is expressed exclusively in hepatocytes, that, in turn, limits systemic exposure.

Longer exposure and repeat dosing will be required to assess the duration of pharmacodynamic effects and further evaluate drug safety. A multiple ascending dose study of SLN360 is underway in subjects with stable ASCVD to further assess its effects (NCT04606602). Although the current study is small in size, the magnitude and duration of effect from single doses is promising and warrants further development.

All documents referenced herein are hereby incorporated by reference in their entirety.

### REFERENCES

1. Boerwinkle E, Leffert C C, Lin J, Lackner C, Chiesa G, and Hobbs H H. Apolipoprotein(a) gene accounts for greater than 90% of the variation in plasma lipoprotein(a) concentrations. J Clin Invest. 1992; 90(1):52-60. doi.org/10.1172/jc1115855
2. Burgess S, Ference B A, Staley J R, Freitag D F, Mason A M, Nielsen S F, Willeit P, Young R, Surendran P, Karthikeyan S, Bolton T R, Peters J E, Kamstrup P R, Tybjmrg-Hansen A, Benn M, Langsted A, Schnohr P, Vedel-Krogh S, Kobylecki C J, Ford I, Packard C, Trompet S, Jukema J W, Sattar N, Di Angelantonio E, Saleheen D, Howson J M M, Nordestgaard B G, Butterworth A S, Danesh J; European Prospective Investigation Into Cancer and Nutrition-Cardiovascular Disease (EPIC-CVD) Consortium. Association of LPA Variants With Risk of Coronary Disease and the Implications for Lipoprotein(a)-Lowering Therapies: A Mendelian Randomization Analysis. JAMA Cardiol. 2018 Jul. 1; 3(7):619-627. doi: 10.1001/jamacardio.2018.1470. PMID: 29926099; PMCID: PMC6481553.
3. Pia R. Kamstrup, MD, Marianne Benn, MD, PhD, Anne Tybjmrg-Hansen, MD, DMSc, and Børge G. Nordestgaard, MD, DMSc. Extreme Lipoprotein(a) Levels and Risk of Myocardial Infarction in the General Population. The Copenhagen City Heart Study Circulation. 2008; 117:176-184.
4. Cybulska B, Klosiewicz-Latoszek L, Penson P E, Banach M. What do we know about the role of lipoprotein(a) in atherogenesis 57 years after its discovery?Prog Cardiovasc Dis. 2020 May-June; 63(3):219-227. doi: 10.1016/j.pcad.2020.04.004. Epub 2020 Apr. 8. PMID: 32277995.
5. P. R. Kamstrup, A. Tybjaerg-Hansen, B. G. Nordestgaard Elevated lipoprotein(a) and risk of aortic valve stenosis in the general population J Am Coll Cardiol, 63 (2014), pp. 470-477
6. R. Capoulade, K. L. Chan, C. Yeang, et al., Oxidized phospholipids, lipoprotein(a), and progression of calcific aortic valve stenosis. J Am Coll Cardiol, 66 (2015), pp. 1236-1246
7. Koschinsky M L, Marcovina S M, May L F, Gabel B R. Analysis of the mechanism of lipoprotein(a) assembly. Clin Genet. 1997 November; 52(5):338-46. doi: 10.1111/j.1399-0004.1997.tb04351.x. PMID: 9520124.
8. Tsimikas S, EKarwatowska-Prokopczuk E, Gouni-Berthold I et al. Lipoprotein(a) Reduction in Persons with Cardiovascular Disease. N Engl J Med 2020; 382:244-255 DOI: 10.1056/NEJMoa1905239.
9. Common Terminology Criteria for Adverse Events (CTCAE) Version 5.0 Published: Nov. 27, 2017 ctep.cancer.gov/protocoldevelopment/electronic_applications/docs/CTCAE_v5_Quick_Ref erence_8.5x11.pdf
10. Guidance for Industry Toxicity Grading Scale for Healthy Adult and Adolescent Volunteers Enrolled in Preventive Vaccine Clinical Trials www.fda.gov/media/73679/download
11. Aaron D. Springer and Steven F. Dowdy. Nucleic Acid Therapeutics. Jun 2018.109-118.http://doi.org/10.1089/nat.2018.0736
12. Mann R, Sekhon S, Sekhon S. Drug-Induced Liver Injury After COVID-19 Vaccine. Cureus. 2021; 13(7):e16491. Published 2021 Jul. 19. doi:10.7759/cureus.16491
13. Sewing S, Roth A B, Winter M, Dieckmann A, Bertinetti-Lapatki C, Tessier Y, et al. (2017) Assessing single-stranded oligonucleotide drug-induced effects in vitro reveals key risk factors for thrombocytopenia. PLoS ONE 12(11): e0187574. doi.org/10.1371/journal.pone.0187574
14. Yeang C, Witztum J L, Tsimikas S. Novel method for quantification of lipoprotein (a)-cholesterol: implications for improving accuracy of LDL-C measurements. J. Lipid Res. (2021) 62 100053
15. Tsimikas S., Brilakis E S, M. D., Miller E R. et al. Oxidized Phospholipids, Lp(a) Lipoprotein, and Coronary Artery Disease. N Engl J Med 2005; 353:46-57 DOI: 10.1056/NEJMoa043175
16. Koren, M. J., Moriarty, P. M., Baum, S. J. et al. Preclinical development and phase 1 trial of a novel siRNA targeting lipoprotein(a). Nat Med (2022). https://doi.org/10.1038/s41591-021-01634-w

1. SLN360 for use in the prophylaxis or treatment of a condition associated with elevated levels of Lp(a), wherein said SLN360 is for administration in an individual dose of 100 mg to 600 mg.

2. SLN360 for use according to claim 1, wherein an individual dose provides SLN360 in an amount of:
100-600 mg, 125-600 mg, 150-600 mg, 175-600 mg, 200-600 mg, 225-600 mg, 250-600 mg, 275-600 mg, 300-600 mg, 325-600 mg, 350-600 mg, 375-600 mg, 400-600 mg, 425-600 mg, 450-600 mg, 475-600 mg, 500-600 mg, 525-600 mg, 550-600 mg or 575-600 mg;
100-575 mg, 125-575 mg, 150-575 mg, 175-575 mg, 200-575 mg, 225-575 mg, 250-575 mg, 275-575 mg, 300-575 mg, 325-575 mg, 350-575 mg, 375-575 mg, 400-575 mg, 425-575 mg, 450-575 mg, 475-575 mg, 500-575 mg, 525-575 mg or 550-575 mg;
100-550 mg, 125-550 mg, 150-550 mg, 175-550 mg, 200-550 mg, 225-550 mg, 250-550 mg, 275-550 mg, 300-550 mg, 325-550 mg, 350-550 mg, 375-550 mg, 400-550 mg, 425-550 mg, 450-550 mg, 475-550 mg, 500-550 mg or 525-550 mg;
100-525 mg, 125-525 mg, 150-525 mg, 175-525 mg, 200-525 mg, 225-525 mg, 250-525 mg, 275-525 mg, 300-525 mg, 325-525 mg, 350-525 mg, 375-525 mg, 400-525 mg, 425-525 mg, 450-525 mg, 475-525 mg or 500-525 mg;
100-500 mg, 125-500 mg, 150-500 mg, 175-500 mg, 200-500 mg, 225-500 mg, 250-500 mg, 275-500 mg, 300-500 mg, 325-500 mg, 350-500 mg, 375-500 mg, 400-500 mg, 425-500 mg, 450-500 mg or 475-500 mg;
100-475 mg, 125-475 mg, 150-475 mg, 175-475 mg, 200-475 mg, 225-475 mg, 250-475 mg, 275-475 mg, 300-475 mg, 325-475 mg, 350-475 mg, 375-475 mg, 400-475 mg, 425-475 mg or 450-475 mg;
100-450 mg, 125-450 mg, 150-450 mg, 175-450 mg, 200-450 mg, 225-450 mg, 250-450 mg, 275-450 mg, 300-450 mg, 325-450 mg, 350-450 mg, 375-450 mg, 400-450 mg or 425-450 mg;
100-425 mg, 125-425 mg, 150-425 mg, 175-425 mg, 200-425 mg, 225-425 mg, 250-425 mg, 275-425 mg, 300-425 mg, 325-425 mg, 350-425 mg, 375-425 mg or 400-425 mg;
100-400 mg, 125-400 mg, 150-400 mg, 175-400 mg, 200-400 mg, 225-400 mg, 250-400 mg, 275-400 mg, 300-400 mg, 325-400 mg, 350-400 mg or 375-400 mg;
100-375 mg, 125-375 mg, 150-375 mg, 175-375 mg, 200-375 mg, 225-375 mg, 250-375 mg, 275-375 mg, 300-375 mg, 325-375 mg or 350-375 mg;
100-350 mg, 125-350 mg, 150-350 mg, 175-350 mg, 200-350 mg, 225-350 mg, 250-350 mg, 275-350 mg, 300-350 mg or 325-350 mg;
100-325 mg, 125-325 mg, 150-325 mg, 175-325 mg, 200-325 mg, 225-325 mg, 250-325 mg, 275-325 mg or 300-325 mg;
100-300 mg, 125-300 mg, 150-300 mg, 175-300 mg, 200-300 mg, 225-300 mg, 250-300 mg or 275-300 mg;
100-275 mg, 125-275 mg, 150-275 mg, 175-275 mg, 200-275 mg, 225-275 mg or 250-275 mg;
100-250 mg, 125-250 mg, 150-250 mg, 175-250 mg, 200-250 mg or 225-250 mg;
100-225 mg, 125-225 mg, 150-225 mg, 175-225 mg or 200-225 mg;
100-200 mg, 125-200 mg, 150-200 mg or 175-200 mg;
100-175 mg, 125-175 mg or 150-175 mg;
100-150 mg or 125-150 mg; or
100-125 mg.

3. SLN360 for use according to claim 1 or claim 2 wherein an individual dose provides SLN360 in an amount of about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg or about 600 mg.

4. SLN360 for use according to any one of claims 1 to 3 wherein an individual dose provides SLN360 in an amount of 300 mg.

5. SLN360 for use according to any one of claims 1 to 3 wherein an individual dose provides SLN360 in an amount of 450 mg.

6. SLN360 for use according to claim 1, wherein said SLN360 is for administration in an individual dose of 300 mg to 600 mg.

7. SLN360 for use according to claim 6, wherein an individual dose provides SLN360 in an amount of:
300-600 mg, 325-600 mg, 350-600 mg, 375-600 mg, 400-600 mg, 425-600 mg, 450-600 mg, 475-600 mg, 500-600 mg, 525-600 mg, 550-600 mg or 575-600 mg;
300-575 mg, 325-575 mg, 350-575 mg, 375-575 mg, 400-575 mg, 425-575 mg, 450-575 mg, 475-575 mg, 500-575 mg, 525-575 mg or 550-575 mg;
300-550 mg, 325-550 mg, 350-550 mg, 375-550 mg, 400-550 mg, 425-550 mg, 450-550 mg, 475-550 mg, 500-550 mg or 525-550 mg;
300-525 mg, 325-525 mg, 350-525 mg, 375-525 mg, 400-525 mg, 425-525 mg, 450-525 mg, 475-525 mg or 500-525 mg;
300-500 mg, 325-500 mg, 350-500 mg, 375-500 mg, 400-500 mg, 425-500 mg, 450-500 mg or 475-500 mg;
300-475 mg, 325-475 mg, 350-475 mg, 375-475 mg, 400-475 mg, 425-475 mg or 450-475 mg;
300-450 mg, 325-450 mg, 350-450 mg, 375-450 mg, 400-450 mg or 425-450 mg;
300-425 mg, 325-425 mg, 350-425 mg, 375-425 mg or 400-425 mg;
300-400 mg, 325-400 mg, 350-400 mg or 375-400 mg;
300-375 mg, 325-375 mg or 350-375 mg;
300-350 mg or 325-350 mg; or
300-325 mg.

8. SLN360 for use according to claim 6 or claim 7 wherein an individual dose provides SLN360 in an amount of about 300 mg, about 325 mg, about 350 mg, about 375 mg, about 400 mg, about 425 mg, about 450 mg, about 475 mg, about 500 mg, about 525 mg, about 550 mg, about 575 mg or about 600 mg.

9. SLN360 for use according to any one of claims 6 to 8 wherein an individual dose provides SLN360 in an amount of 300 mg.

10. SLN360 for use according to any one of claims 6 to 8 wherein an individual dose provides SLN360 in an amount of 450 mg.

11. SLN360 for use according to any one of claims 1 to 10 wherein SLN360 is administered to a subject via a dosing regime comprising a plurality or course of doses.

12. SLN360 for use according to claim 11 wherein administration is monthly, 2-monthly, 3-monthly, 4-monthly, 5-monthly, 6-monthly, 7-monthly, 8-monthly, 9-monthly, 10-monthly, 11-monthly or 12-monthly.

13. SLN360 for use according to claim 11 or claim 12 wherein administration is 4-monthly.

14. SLN360 for use according to claim 11 or claim 12 wherein administration is 6-monthly.

15. SLN360 for use according to claim 11 or claim 12 wherein an individual dose provides SLN360 in an amount of 300 mg and administration is 4-monthly.

16. SLN360 for use according to claim 11 or claim 12 wherein an individual dose provides SLN360 in an amount of 300 mg and administration is 6-monthly.

17. SLN360 for use according to claim 11 or claim 12 wherein an individual dose provides SLN360 in an amount of 450 mg and administration is 6-monthly.

18. SLN360 for use according to claim 11 or claim 12 wherein the interval between consecutive doses is at least 1 month, at least 2 months, at least 3 months, at least 4 months, at least 5 months, at least 6 months, at least 7 months, at least 8 months, at least 9 months, at least 10 months, at least 11 months, or at least 12 months.

19. SLN360 for use according to claim 11 or claim 12 wherein the interval between consecutive doses is at least 4 months.

20. SLN360 for use according to claim 11 or claim 12 wherein the interval between consecutive doses is at least 6 months.

21. SLN360 for use according to claim 11 or claim 12 wherein an individual dose provides SLN360 in an amount of 300 mg and the interval between consecutive doses is at least 4 months.

22. SLN360 for use according to claim 11 or claim 12 wherein an individual dose provides SLN360 in an amount of 300 mg and the interval between consecutive doses is at least 6 months.

23. SLN360 for use according to claim 11 or claim 12 wherein an individual dose provides SLN360 in an amount of 450 mg and the interval between consecutive doses is at least 6 months.

24. SLN360 for use according to any one of the preceding claims wherein the SLN360 is administered by subcutaneous injection.

25. SLN360 for use according to any one of the preceding claims wherein the condition associated with elevated levels of Lp(a) is cardiovascular disease.

26. SLN360 for use according to claim 25 wherein the condition associated with elevated levels of Lp(a) is atherosclerotic cardiovascular disease or atherosclerotic cerebrovascular disease

27. SLN360 for use according to any one of the preceding claims wherein the condition associated with elevated levels of Lp(a)-containing particles is stroke (e.g., ischaemic stroke), atherosclerosis, thrombosis, coronary heart disease, aortic stenosis (e.g., calcific aortic stenosis), hyperlipidaemia, dyslipidaemia, coronary artery disease, peripheral arterial disease, abdominal aortic aneurysm, heart failure (e.g., heart failure secondary to ischaemic cardiomyopathy), or familial hypercholesterolaemia.

28. SLN360 for use according to any one of claims 1 to 24 wherein the condition associated with elevated levels of Lp(a) is a cancer.

29. SLN360 for use according to claim 28 wherein the cancer is prostate cancer.
